(19) United States
(12) Patent Application Publication
(10) Pub. No.: US 2022/0117646 A1
(43) Pub. Date: Apr. 21, 2022

(54) TELESCOPING SMOKE EVACUATION DEVICE FOR USE WITH HANDHELD SURGICAL INSTRUMENT

(71) Applicant: Pathy Medical, LLC, Shelton, CT (US)

(72) Inventors: Gennady Kleyman, Brooklyn, NY (US); Mikiya Silver, New Haven, CT (US)

(73) Assignee: Pathy Medical, LLC, Shelton, CT (US)

(21) Appl. No.: 17/072,331

(22) Filed: Oct. 16, 2020

(51) Int. Cl. A61B 18/12

(52) U.S. Cl. CPC A61B 18/12; A61B 2018/00916; A61B 2218/008

(57) ABSTRACT

A smoke evacuation device is disclosed for use with a handheld surgical instrument, which includes an elongated tubular body having opposed proximal and distal end portions, wherein the proximal end portion of the tubular body is adapted and configured for communicating with a source of suction and the distal end portion of the tubular body is adapted and configured to intake smoke generated at a surgical site, and attachment means for attaching the tubular body to the handheld surgical instrument.

### BACKGROUND OF THE INVENTION

### 1. Field of the Invention

The subject invention is directed to surgical instrumentation, and more particularly, to a telescoping smoke evacuation device that is configured for attachment to a handheld electrosurgical instrument.

### 2. Description of Related Art

In medical practice, a handheld electrosurgical device, such as a BOVIE® pencil, is typically used to incise tissue during a surgical procedure. The use of a handheld electrosurgical device, such as a BOVIE® pencil to incise tissue, typically creates surgical smoke when energy is imparted to tissue cells during surgery. The heat from the energy vaporizes the intracellular fluid, which increases the pressure inside the cell and eventually causes the cell membrane to burst. When this happens, a plume of smoke containing mostly water vapor is released into the operating room. At the same time, the heat chars the protein and other organic matter within the cell, releasing contaminants, such as carbonized cell fragments and gaseous hydrocarbons.

These small particles and gases are potentially hazardous if inhaled. If they are not evacuated from the surgical site nearest to the location where the surgical smoke is created, they can become airborne and inhaled. This has led to the development and use of smoke evacuation systems during surgical procedures. A smoke evacuator is basically a vacuum pump with one or more filters designed to remove surgical smoke and aerosol from an operative site and filter out contaminants. In addition, they may return filtered air to the operating room.

Given the increasing need and desire for surgical evacuation systems in operating rooms, the inventors have determined that it would be advantageous to develop a telescoping smoke evacuation device that can be readily attached to a handheld electrosurgical instrument to intake smoke generated at a surgical site, and which can be readily extended and retracted relative to an electrocautery blade/end effector to allow for improved suction at the site where smoke is generated while providing flexibility to avoid anatomy when in a tight surgical pocket, as well as the ability to match the length of the electrocautery blade/end effector.

### SUMMARY OF THE DISCLOSURE

The subject invention is directed to a new and useful smoke evacuation device for use with a handheld surgical instrument, which includes an elongated tubular body having opposed proximal and distal end portions, wherein the proximal end portion of the tubular body is adapted and configured for communicating with a source of suction and the distal end portion of the tubular body is adapted and configured to intake smoke generated at a surgical site, and distal attachment means for attaching the distal end portion of the tubular body adjacent to a distal end portion of the handheld surgical instrument. Preferably, the distal attachment means is adapted and configured to maintain a predetermined ergonomic distance between the tubular body and the handheld surgical instrument.

In an embodiment of the invention, the distal attachment means includes a strap of adhesive material, a hook and loop type fastener strap or a cable tie strap. In another embodiment of the invention, means includes a spring biased compression clamp with compliant contact surfaces. In yet another embodiment of the invention, the distal attachment means includes a pair of spaced apart deflectable arms with compliant contact surfaces. In still another embodiment of the invention, the distal attachment means includes a flexible snap-on curved compression clasp. In another embodiment of the invention, the distal attachment means includes a clip for engaging an end effector extending from the distal end portion of the handheld surgical instrument.

Alternatively, the distal attachment means includes a mounting block that is adapted and configured for spring biased compressive engagement on the distal end portion of the handheld surgical instrument, which includes a rail on a side wall thereof for slidably engaging with an elongated track extending along the tubular body. Here, a pinion gear can be included on the side wall of the mounting block for cooperating with a gear rack provided adjacent the elongated track for advancing or retracting the tubular body relative to the distal end portion of the handheld surgical instrument.

In another embodiment of the invention, the distal attachment means includes a mounting sleeve that is adapted and configured for surrounding engagement on the distal end portion of the handheld surgical instrument, wherein the mounting sleeve includes an outer body portion and an inner body portion that is mounted to extend from and retract into the outer body portion to accommodate end effectors of differing length. An annular smoke evacuation passage is formed between the outer body portion and the inner body portion of the mounting sleeve.

In accordance with another embodiment of the invention, the smoke evacuation device further includes proximal attachment means for attaching the proximal end portion of the tubular body adjacent to a proximal end portion of the handheld surgical instrument. In an embodiment of the invention, the proximal attachment means includes a clip for engaging a power cord extending from the proximal end portion of the handheld surgical instrument. In another embodiment of the invention, the proximal attachment means incudes a pair of spaced apart deflectable arms with compliant contact surfaces. In yet another embodiment of the invention, the proximal attachment means incudes a flexible snap-on curved compression clasp.

In accordance with an embodiment of the invention, the tubular body includes a telescoping tube that is adapted and configured to extend from and retract into the distal end of the tubular body. In one embodiment the telescoping tube and the tubular body are threadably associated with one another. In another embodiment, a proximal end of the telescoping tube and the distal end of the tubular body are sealingly associated with one another. In yet another embodiment of the invention, the tubular body has an offset section between the opposed proximal and distal end portions thereof. In still another embodiment of the invention, a nozzle tip is operatively associated with a distal end of the telescoping tube. In some embodiments of the invention, the nozzle tip has a tapered conical construction, and in others the nozzle tip has an intake portion that surrounds an end effector extending from the distal end portion of the handheld surgical instrument.

In sum, a smoke evacuation device is disclosed for use with a handheld electrosurgical instrument, which includes an elongated tubular body having opposed proximal and distal end portions, and either distal attachment means for attaching the distal end portion of the tubular body adjacent to a distal end portion of the handheld surgical instrument, proximal attachment means for attaching the proximal end portion of the tubular body adjacent to a proximal end portion of the handheld surgical instrument, or both distal and proximal attachment means for attaching the distal and proximal end portions of the tubular body adjacent to distal and proximal end portions of the handheld surgical instrument, respectively.

These and other features of the smoke evacuation devices of the subject invention will become more readily apparent to those having ordinary skill in the art to which the subject invention appertains from the detailed description of the preferred embodiments taken in conjunction with the following brief description of the drawings.

### BRIEF DESCRIPTION OF THE DRAWINGS

So that those skilled in the art will readily understand how to make and use the lighting device of the subject invention without undue experimentation, preferred embodiments thereof will be described in detail herein below with reference to the figures wherein:

FIG. 1 is an isometric view of a smoke evacuation telescopic attachment and adhesive tape before attached to the electrosurgical instrument;

FIGS. 2 and 3 are isometric views of a smoke evacuation telescopic attachment attached to the electrosurgical instrument by adhesive tape;

FIG. 4 is an isometric view of a smoke evacuation telescopic attachment attached to another side of the electrosurgical instrument by adhesive tape;

FIGS. 5 through 7 are isometric view of a smoke evacuation telescopic attachment attached to the electrosurgical instrument by adhesive tape and suction tubing adjusted to different length and type of electrodes;

FIG. 8 is a front view of a smoke evacuation telescopic attachment attached to the electrosurgical instrument;

FIG. 8A is a partial top view of a smoke evacuation telescopic attachment attached to the electrosurgical instrument;

FIG. 9 is an exploded isometric view of a smoke evacuation telescopic attachment;

FIG. 10 is an isometric view of a smoke evacuation telescopic attachment;

FIGS. 11 and 12 are front and side views of a smoke evacuation telescopic attachment;

FIG. 13 is a cross-section taken along line A-A of FIG. 12;

FIGS. 14 through 17 are views of suction tubing;

FIG. 18 is a cross-section taken along line B-B of FIG. 17;

FIG. 19 is an isometric view of a smoke evacuation telescopic attachment before being attached to the electrosurgical instrument;

FIG. 20 is an isometric view of a smoke evacuation telescopic attachment attached to the electrosurgical instrument;

FIG. 21 is an isometric view of a smoke evacuation telescopic attachment attached to another side of the electrosurgical instrument;

FIGS. 22 through 24 are isometric views of a smoke evacuation telescopic attachment attached to the electrosurgical instrument suction tubing adjusted to different length and type of electrodes;

FIG. 25 is a front view of a smoke evacuation telescopic attachment attached to the electrosurgical instrument;

FIG. 26 is a partial top view of a smoke evacuation telescopic attachment attached to the electrosurgical instrument;

FIG. 27 is a side view of a smoke evacuation telescopic attachment attached to the electrosurgical instrument;

FIG. 28 is a partial bottom view of a smoke evacuation telescopic attachment attached to the electrosurgical instrument;

FIG. 29 is a cross-section taken along line C-C of FIG. 26;

FIG. 30 is an isometric view of a smoke evacuation telescopic attachment in a compact retracted state;

FIG. 31 is a front view of a smoke evacuation telescopic attachment in a compact retracted state;

FIG. 32 is a partial side view of a smoke evacuation telescopic attachment in a compact retracted state;

FIGS. 33 and 34 are partial side and top views of a smoke evacuation telescopic attachment in a compact retracted state;

FIG. 35 is an exploded perspective view of a smoke evacuation telescopic attachment;

FIG. 36 is a cross-section take along line D-D of FIG. 34;

FIG. 37 is an isometric view of a smoke evacuation telescopic attachment in an extended state;

FIG. 38 is a side view of a smoke evacuation telescopic attachment in extended state;

FIG. 39 is a cross-section taken along line E-E of FIG. 38;

FIG. 40 is an enlarged view of area F of FIG. 39;

FIG. 41 is an enlarged view of area G of FIG. 39;

FIG. 42 is an isometric view of a clamp;

FIGS. 43 and 44 are front and side views of the clamp;

FIG. 45 is an isometric view of another smoke evacuation telescopic attachment before it is attached to the electrosurgical instrument;

FIG. 46 is an isometric view of the smoke evacuation telescopic attachment attached to the electrosurgical instrument;

FIG. 47 is an isometric view (from another perspective) of a smoke evacuation telescopic attachment attached to the electrosurgical instrument;

FIG. 48 is an isometric view of a smoke evacuation telescopic attachment attached to another side of the electrosurgical instrument;

FIGS. 49 and 50 are isometric views of a smoke evacuation telescopic attachment attached to the electrosurgical instrument suction tubing adjusted to different length and type of electrodes;

FIG. 51 is a front view of smoke evacuation telescopic attachment attached to the electrosurgical instrument;

FIGS. 52 and 53 are two side views of a smoke evacuation telescopic attachment attached to the electrosurgical instrument;

FIGS. 54 and 55 are front and back views of a smoke evacuation telescopic attachment attached to the electrosurgical instrument

FIG. 56 is an isometric view of a smoke evacuation telescopic attachment in a retracted compact state;

FIG. 57 is an exploded view of a smoke evacuation telescopic attachment;

FIG. 58 is an enlarged view of area H of FIG. 57;

FIG. 59 is an enlarged view of area J of FIG. 57;

FIGS. 60 and 61 are side and top views of a smoke evacuation telescopic attachment in compact retracted state;

FIG. 62 is a cross-section taken along line K-K of FIG. 60;

FIG. 63 is a side view of a smoke evacuation telescopic attachment in extended state;

FIG. 64 is a cross-section taken along line L-L of FIG. 63;

FIG. 65 is an enlarged view of area M of FIG. 64;

FIG. 66 is an enlarged view of area N of FIG. 64;

FIG. 67 is an isometric view of outside tubing;

FIG. 68 is a side view of outside tubing;

FIGS. 69 and 70 are front and back views of outside tubing;

FIGS. 71 and 72 are top and isometric views of a compliant (rubber like) plate;

FIG. 73 is an isometric view of a smoke evacuation telescopic attachment before attached to the electrosurgical instrument;

FIG. 74 is an isometric view of a smoke evacuation telescopic attachment attached to the electrosurgical instrument;

FIG. 75 is an isometric view of a smoke evacuation telescopic attachment attached to another side of the electrosurgical instrument;

FIGS. 76 through 78 are isometric views of a smoke evacuation telescopic attachment attached to the electrosurgical instrument and suction tubing adjusted to different length and type of electrodes;

FIG. 79 is a top view of a smoke evacuation telescopic attachment attached to the electrosurgical instrument;

FIG. 80 is a side view of a smoke evacuation telescopic attachment attached to the electrosurgical instrument;

FIGS. 81 and 82 are front and back views of a smoke evacuation telescopic attachment attached to the electrosurgical instrument;

FIG. 83 is an isometric view of a smoke evacuation telescopic attachment in a compact retracted state;

FIG. 84 is an exploded view of a smoke evacuation telescopic attachment;

FIG. 85 is an enlarged view of area P of FIG. 84;

FIG. 86 is an enlarged view of area Q of FIG. 84;

FIG. 87 is an isometric view of outside tubing;

FIGS. 88 and 89 are front and back views of outside tubing;

FIG. 90 is an isometric view before a holding device and a smoke evacuation telescopic attachment are attached to the electrosurgical instrument;

FIG. 91 is an isometric view the holding device attached to the electrosurgical instrument;

FIG. 92 is an isometric view of the holding device attached to the electrosurgical instrument and the telescopic attachment attached to the holding device;

FIG. 93 is an isometric view of the holding device attached to the electrosurgical instrument and the telescopic attachment attached to another side of the holding device;

FIGS. 94 and 95 are isometric views of a smoke evacuation telescopic attachment attached to the holding device and suction tubing adjusted to different length and type of electrodes;

FIGS. 96 and 97 are front and back views of the holding device attached to the electrosurgical instrument and telescopic attachment attached to another side of the holding device;

FIG. 96 and FIG. 97 are front and back views of the holding device attached to the electrosurgical instrument and the telescopic attachment attached to another side of the holding device;

FIGS. 98 through 100 are top, side and bottom views of the holding device attached to the electrosurgical instrument and telescopic attachment attached to another side of the holding device;

FIG. 101 is a cross-section taken along line R-R of FIG. 98;

FIG. 102 is an exploded perspective view of the holding device;

FIGS. 103 and 104 are perspective views of the holding device;

FIGS. 105 and 106 are front and side views of the holding device;

FIG. 107 is a cross-section taken along line S-S of FIG. 105;

FIGS. 108 and 109 are perspective views of the outside body;

FIGS. 110 and 111 are perspective views of an inside ring;

FIG. 112 is a perspective view of the cover;

FIG. 113 is an isometric exploded view of the telescopic attachment;

FIG. 114 is an isometric view of the telescopic attachment;

FIG. 115 is a front view of the sliding tubing;

FIG. 116 is a side view of the sliding tubing;

FIG. 117 is a cross-section taken along line T-T of FIG. 116;

FIG. 118 is an isometric view before the holding device and smoke evacuation telescopic attachment are attached to the electrosurgical instrument;

FIG. 119 is an isometric view of the holding device attached to the electrosurgical instrument;

FIG. 120 is an isometric view of a holding device attached to the electrosurgical instrument and telescopic attachment attached to the holding device;

FIG. 121 is an isometric view of the holding device attached to the electrosurgical instrument and telescopic attachment attached to another side of the holding device;

FIGS. 122 and 123 are isometric views of a smoke evacuation telescopic attachment attached to the holding device and suction tubing adjusted to different length and type of electrodes;

FIG. 124 is an enlarged view or area U of FIG. 123;

FIGS. 125 through 127 are top, left and right side views of the view holding device attached to the electrosurgical instrument and telescopic attachment attached to the holding device;

FIGS. 128 and 129 are front and back views of view holding device attached to the electrosurgical instrument and telescopic attachment attached to the holding device;

FIG. 130 is a cross-section taken along line V-V of FIG. 125;

FIG. 131 is an isometric view of a holding device;

FIG. 132 is an exploded perspective view of the holding device;

FIGS. 133 and 134 are front and back views of the holding device;

FIG. 135 is a perspective view of the holding device outside body;

FIGS. 136 through 138 are front, side and top side views of the view holding device outside body;

FIG. 139 is an isometric exploded view of the telescopic attachment;

FIG. 140 is an isometric view of the telescopic attachment;

FIG. 141 is an isometric view of the tubing body with teeth;

FIGS. 142 through 144 are front, right and left side views of the tubing body;

FIG. 145 is a cross-section taken along line W-W of FIG. 143;

FIG. 146 is an isometric view of another smoke evacuation telescopic attachment before it is attached to the electrosurgical instrument;

FIG. 147 is an isometric view of the smoke evacuation telescopic attachment attached to the electrosurgical instrument;

FIG. 148 is an isometric view of the smoke evacuation telescopic attachment attached to another side of the electrosurgical instrument;

FIGS. 149 through 151 are isometric views of the smoke evacuation telescopic attachment attached to the electrosurgical instrument and suction tubing adjusted to different length and type of electrodes;

FIGS. 152 and 153 are front and back views of a telescopic attachment attached to electrosurgical device;

FIGS. 154 through 156 are top, side and bottom views of the telescopic attachment attached to electrosurgical device;

FIG. 157 is a cross-section taken along line Y-Y of FIG. 154;

FIG. 158 is a cross-section taken along line X-X of FIG. 155;

FIG. 159 is an exploded isometric view of a smoke evacuation telescopic attachment;

FIG. 160 is an isometric view of a smoke evacuation telescopic attachment;

FIG. 161 is an enlarged view of area Z of FIG. 158;

FIG. 162 is a back view of a smoke evacuation telescopic attachment;

FIG. 163 is a front view of a smoke evacuation telescopic attachment;

FIG. 164 is a cross-section taken along line AA-AA of FIG. 163;

FIGS. 165 and 166 are side and top views of a smoke evacuation telescopic attachment;

FIG. 167 is a cross-section taken along line AB-AB of FIG. 166;

FIG. 168 is a cross-section taken along line AC-AC of FIG. 166;

FIG. 169 is an exploded perspective view of a telescopic attachment tip assembly;

FIG. 170 is an isometric view of a telescopic attachment tip assembly;

FIGS. 171 through 173 are front, side and back views of a telescopic attachment tip assembly;

FIG. 174 is a cross-section taken along line AD-AD of FIG. 171;

FIG. 175 is an isometric view of another smoke evacuation telescopic attachment before it is attached to the electrosurgical instrument;

FIG. 176 is an isometric view of a smoke evacuation telescopic attachment attached to the electrosurgical instrument;

FIG. 177 is an isometric view of a smoke evacuation telescopic attachment attached to another side of the electrosurgical instrument;

FIGS. 178 and 179 are isometric views of the smoke evacuation telescopic attachment attached to the electrosurgical instrument before and after the smoke evacuation tip is adjusted to the first type of electrode;

FIGS. 180 and 181 are isometric views of the smoke evacuation telescopic attachment attached to the electrosurgical instrument before and after the smoke evacuation tip adjusted to the second type of electrode;

FIGS. 182 and 183 are front and back views of a telescopic attachment attached to the electrosurgical device;

FIGS. 184 and 185 are top and side views of a telescopic attachment attached to the electrosurgical device;

FIG. 186 is a cross-section taken along line AE-AE of FIG. 184;

FIG. 187 is a cross-section taken along line AF-AF of FIG. 185;

FIG. 188 is an exploded isometric view of a smoke evacuation telescopic attachment;

FIG. 189 is an isometric view of a smoke evacuation telescopic attachment;

FIGS. 190 and 191 are front and back views of a smoke evacuation telescopic attachment;

FIG. 192 is a side view of the smoke evacuation telescopic attachment in an initial compact state;

FIG. 193 is a cross-section taken along line AG-AG of FIG. 192;

FIG. 194 are side and back views of the smoke evacuation telescopic attachment in the extended and adjusted state;

FIG. 195 is a cross-section taken along line AH-AH of FIG. 194;

FIG. 196 is a perspective view of the attachment outside body;

FIGS. 197 and 198 are front and back views of the attachment outside body;

FIG. 199 is a side view of the attachment outside body;

FIG. 200 is a cross-section taken along line AJ-AJ of FIG. 199;

FIGS. 201 and 202 are perspective views of a suction tip;

FIGS. 203 through 205 are front, side and back views of the suction tip

FIG. 206 is a cross-section taken along line AK-AK of FIG. 204;

FIG. 207 is an isometric view of a smoke evacuation telescopic attachment before attached to the electrosurgical instrument;

FIG. 208 is an isometric view of a smoke evacuation telescopic attachment attached to the electrosurgical instrument;

FIG. 209 is an isometric view of a smoke evacuation telescopic attachment attached to another side of the electrosurgical instrument;

FIGS. 210 through 212 are isometric views of a smoke evacuation telescopic attachment attached to the electrosurgical instrument and suction tubing adjusted to different length and type of electrodes;

FIGS. 213 through 215 are front, back and top views of a smoke evacuation telescopic attachment attached to the electrosurgical instrument;

FIGS. 216 and 217 are side views of a smoke evacuation telescopic attachment attached to the electrosurgical instrument;

FIG. 218 is an enlarged view of area AL of FIG. 217;

FIG. 219 is an enlarged view of area AM of FIG. 217;

FIG. 220 is an exploded isometric view of a smoke evacuation telescopic attachment;

FIG. 221 is an isometric view of a smoke evacuation telescopic attachment;

FIGS. 222 and 223 are front and back views of a smoke evacuation telescopic attachment;

FIGS. 224 and 225 are side and top views of a smoke evacuation telescopic attachment;

FIG. 226 is a cross-section taken along line AN-AN of FIG. 225;

FIG. 227 and FIG. 228—is a front and back views of smoke evacuation attachment outside body

FIG. 229 and FIG. 230—is a side and top views of smoke evacuation attachment outside body

FIG. 231 is an isometric view of another smoke evacuation telescopic attachment before attached to the electrosurgical instrument;

FIG. 232 is an isometric view of the smoke evacuation telescopic attachment attached to the electrosurgical instrument;

FIG. 233 is an isometric view of the smoke evacuation telescopic attachment attached to another side of the electrosurgical instrument;

FIGS. 234 and 235 are isometric views of the smoke evacuation telescopic attachment attached to the electrosurgical instrument and suction tubing adjusted to different suction points;

FIGS. 236 and 237 are front and back views of a smoke evacuation telescopic attachment attached to the electrosurgical instrument;

FIGS. 238 and 239 are side and top views of a smoke evacuation telescopic attachment attached to the electrosurgical instrument;

FIG. 240 is an exploded isometric view of a smoke evacuation telescopic attachment;

FIGS. 241 and 242 are front and back views of a smoke evacuation telescopic attachment;

FIGS. 243 and 244 are top and side views of a smoke evacuation telescopic attachment;

FIG. 245 is a cross-section taken along line AP-AP of FIG. 243;

FIG. 246 is a cross-section taken along line AR-AR of FIG. 244;

FIG. 247 is an isometric view of a smoke evacuation telescopic attachment outside body;

FIG. 248 is an isometric view of a smoke evacuation telescopic attachment inside tube; and

FIGS. 249 and 250 are perspective view of the tip.

### DETAILED DESCRIPTION OF THE PREFERRED EMBODIMENTS

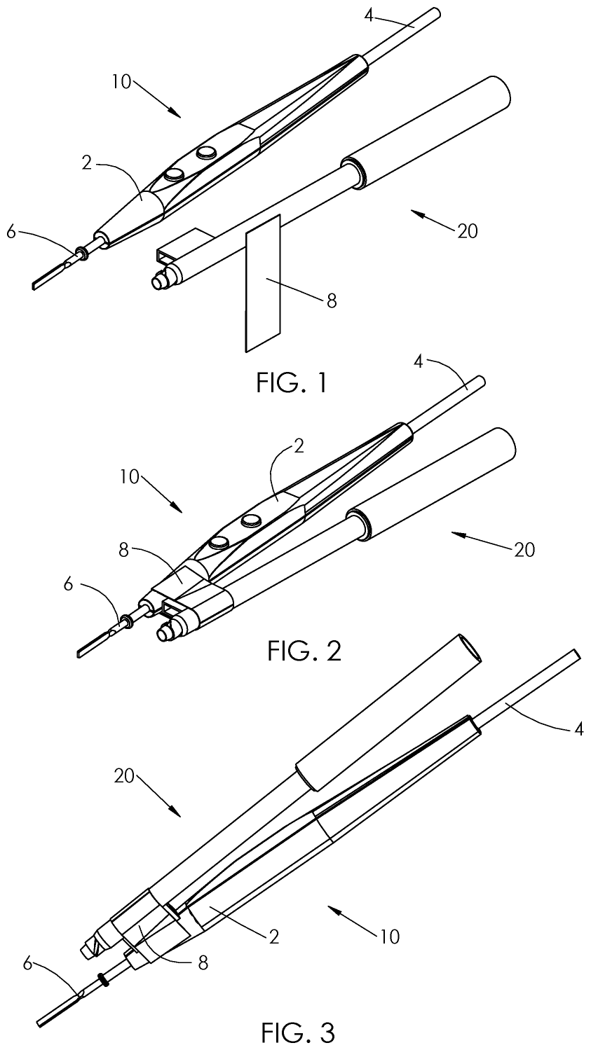

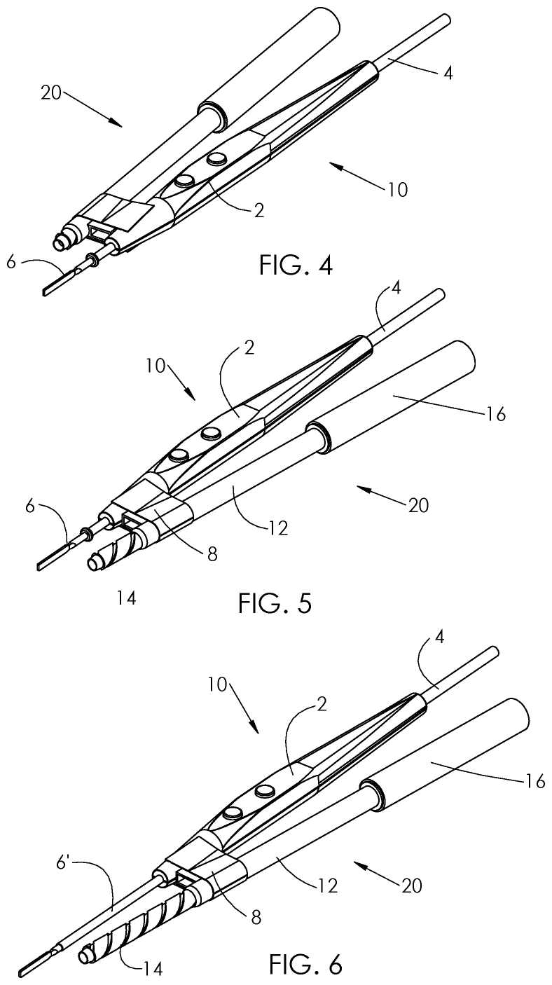

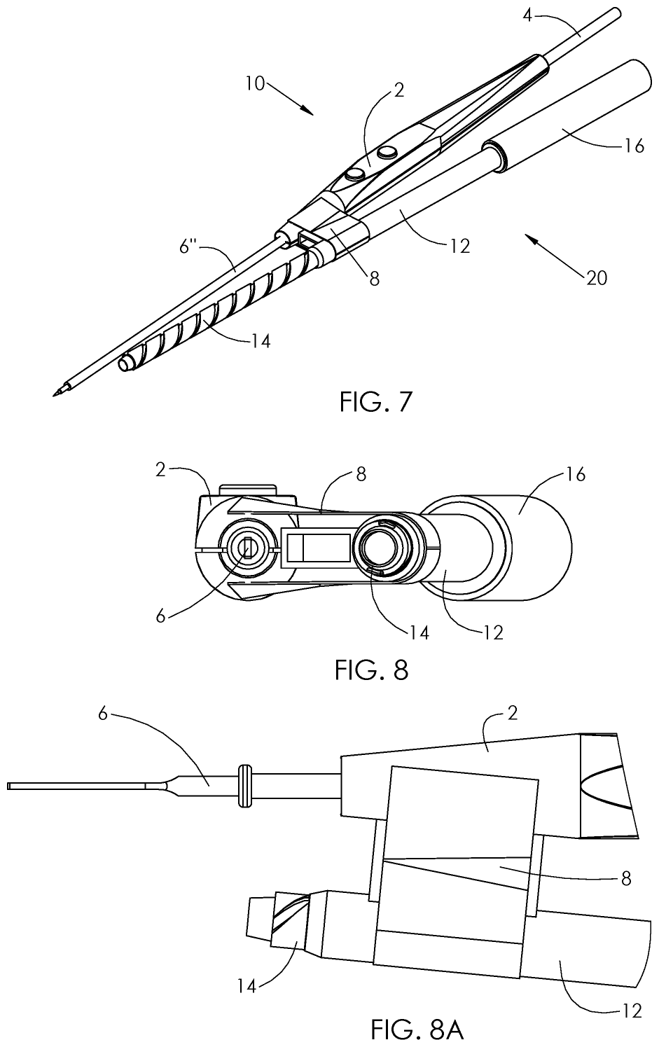

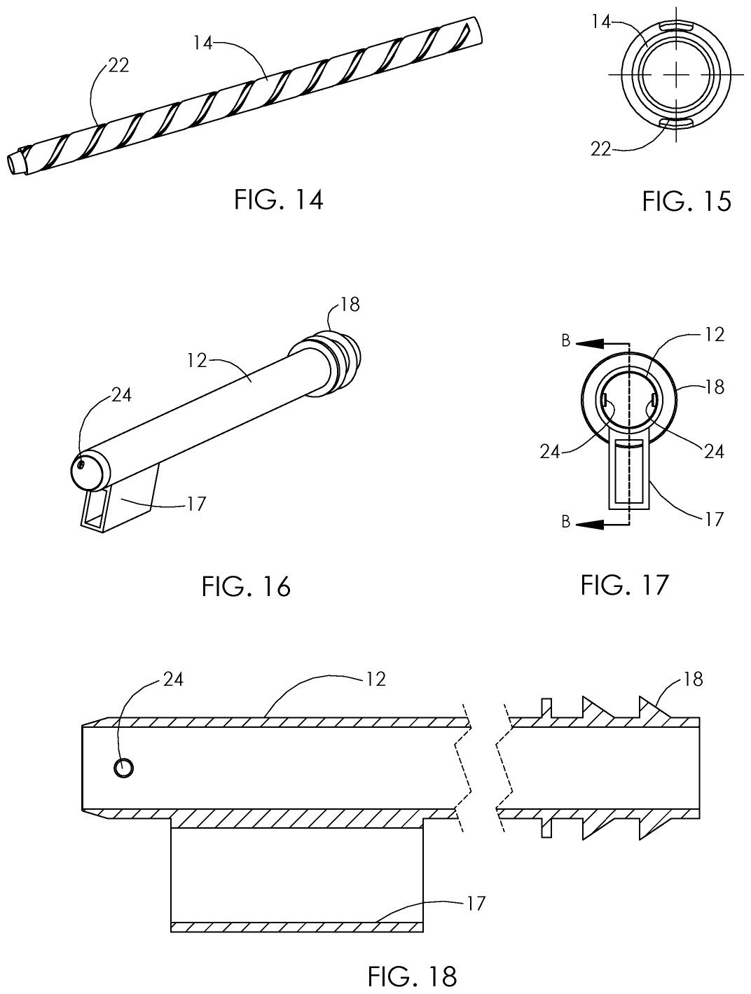

Referring now to the drawings wherein like reference numerals identify similar structural elements of the various embodiments of the subject invention, there is illustrated in FIGS. 1 through 18, a new and useful smoke evacuation device 20 for use with a handheld electrosurgical instrument 10 having an elongated body 2. An electrical cable 4 extends from the proximal end of the body 2 for attachment to an electrical power source and an end effector 6 in the form of an electrocautery blade extends from the distal end of the body 2 for cauterizing and/or incising tissue.

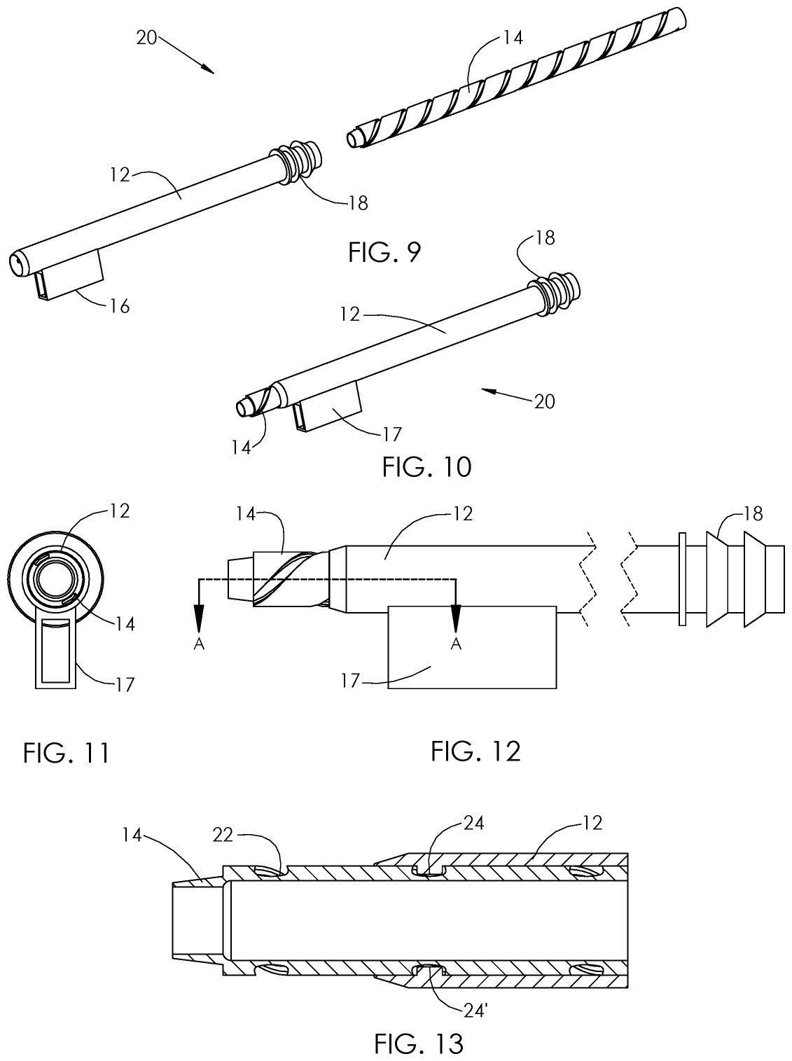

The smoke evacuation device 20 includes an elongated tubular body having opposed proximal and distal body portions, wherein the proximal body portion 16 is adapted and configured for communicating with a source of suction and the distal body portion 12 is adapted and configured to intake and removing smoke generated at a surgical site. The distal body portion 12 includes a proximal sealing barb 18 (see FIGS. 9 through 12, 16 and 18) for sealing engagement within the distal end of the proximal body portion 16.

A distal attachment feature 8 is provided for attaching the distal body portion 12 of smoke evacuation device 20 adjacent to a distal end portion of the surgical instrument 10. Preferably, as illustrated in FIGS. 9 through 12 and FIGS. 16 through 18, the distal attachment feature 8 is operatively associated with a laterally extending spacer flange 17 that is adapted and configured to maintain a predetermined ergonomic distance between the tubular body of device 20 and the handheld surgical instrument 10, as best seen in FIGS. 8 and 8A.

In an embodiment if the invention, the distal attachment feature 8 can be in the form of a strap of adhesive material, a hook and loop type fastener strap or a cable tie strap. A user can place the smoke evacuation device 20 at an appropriate location or distance and then secure the strap or attach the adhesive to the body 2 of surgical instrument 10. The user can then remove the strap or break the adhesive and adjust the position of the device and then reattach it.

The smoke evacuation device 20 further includes a telescoping tube 14 that is adapted and configured to extend from and retract into the distal end of the distal body portion 12. The telescoping tube 14 and the distal body portion 12 are threadably associated with one another, where radially inwardly extending cylindrical bosses 24 are provided within the bore of distal body portion 12 (see FIGS. 16 through 18) for cooperative engagement with a helical groove 22 formed in the exterior surface of telescoping tube 14, as best seen in FIGS. 11 through 13. The telescoping tube 14 advances distally and retracts proximally by axially rotating the tube 14 relative to the distal body portion 12.

Those skilled in the art will readily appreciate that the telescoping tube 14 can be readily extended and retracted relative to the electrocautery blade/end effector 6 to allow for improved suction at the site where smoke is generated while providing flexibility to avoid anatomy when in a tight surgical pocket, as well as the ability to match the length of the electrocautery blade/end effector, as shown for example in FIGS. 5 through 7, with respect to the three end effectors 6, 6′ and 6″.

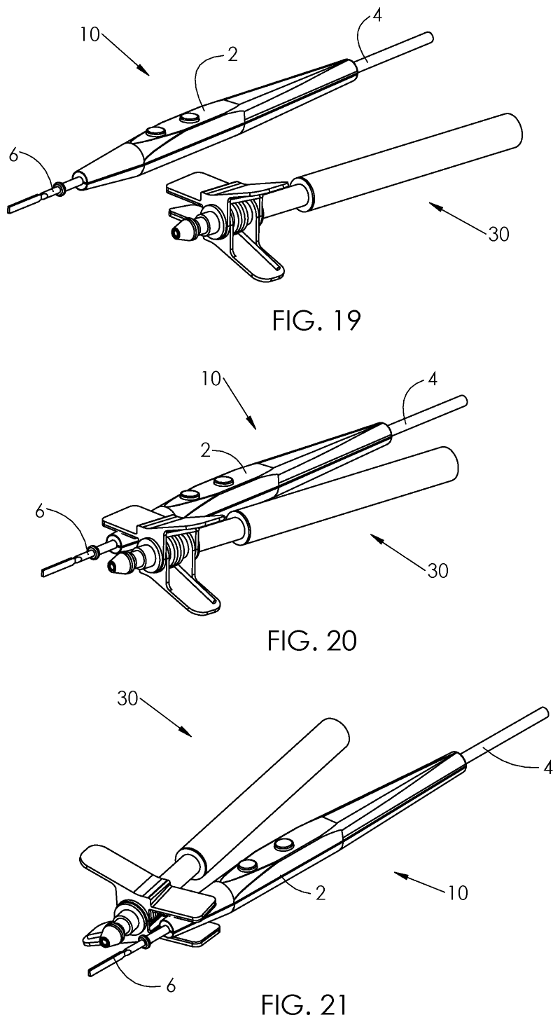

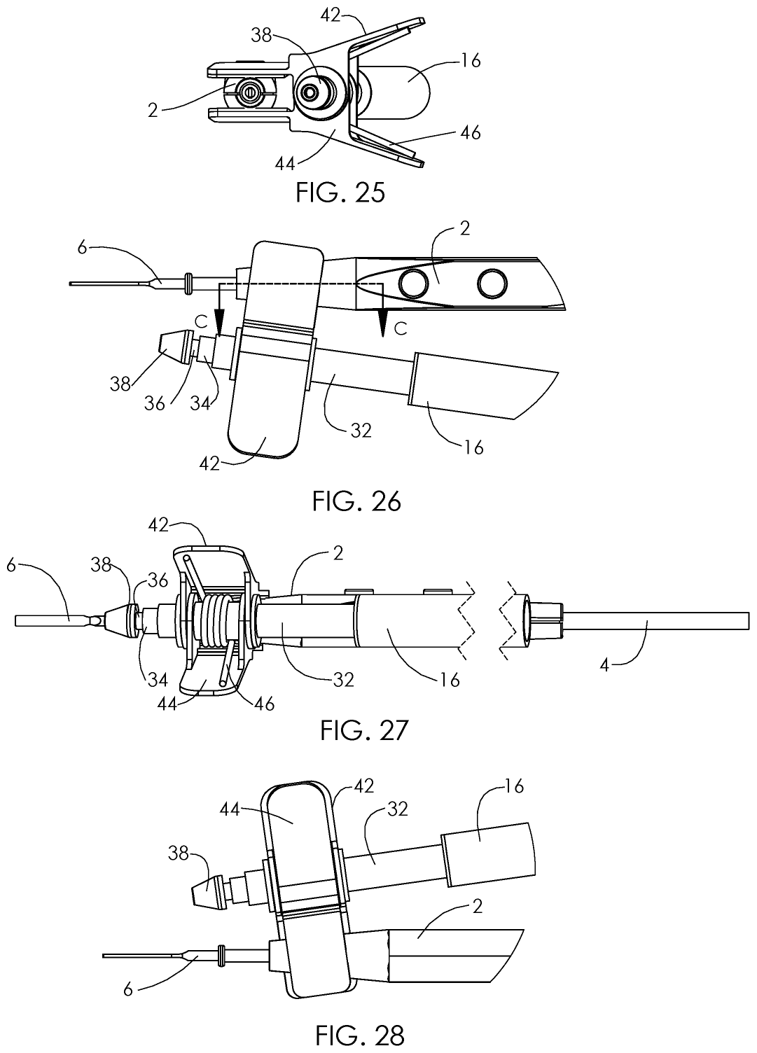

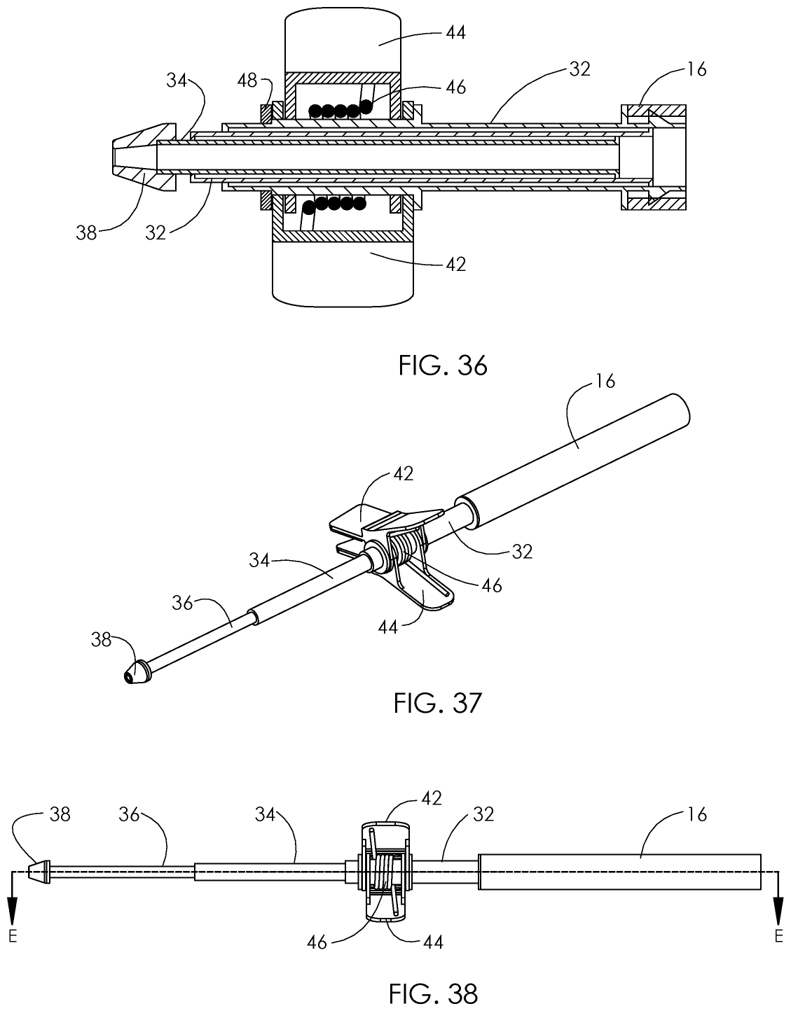

Referring now to FIGS. 19 through 44, there is illustrated another new and useful smoke evacuation device 30 for use with a handheld electrosurgical instrument 10 having an elongated body 2. In this embodiment of the invention, the smoke evacuation device 30 includes a proximal body portion 16 and a distal body portion 32. As best seen in FIG. 35, the proximal end of the distal body portion 32 includes a sealing barb for engagement in the distal end of proximal body portion 16.

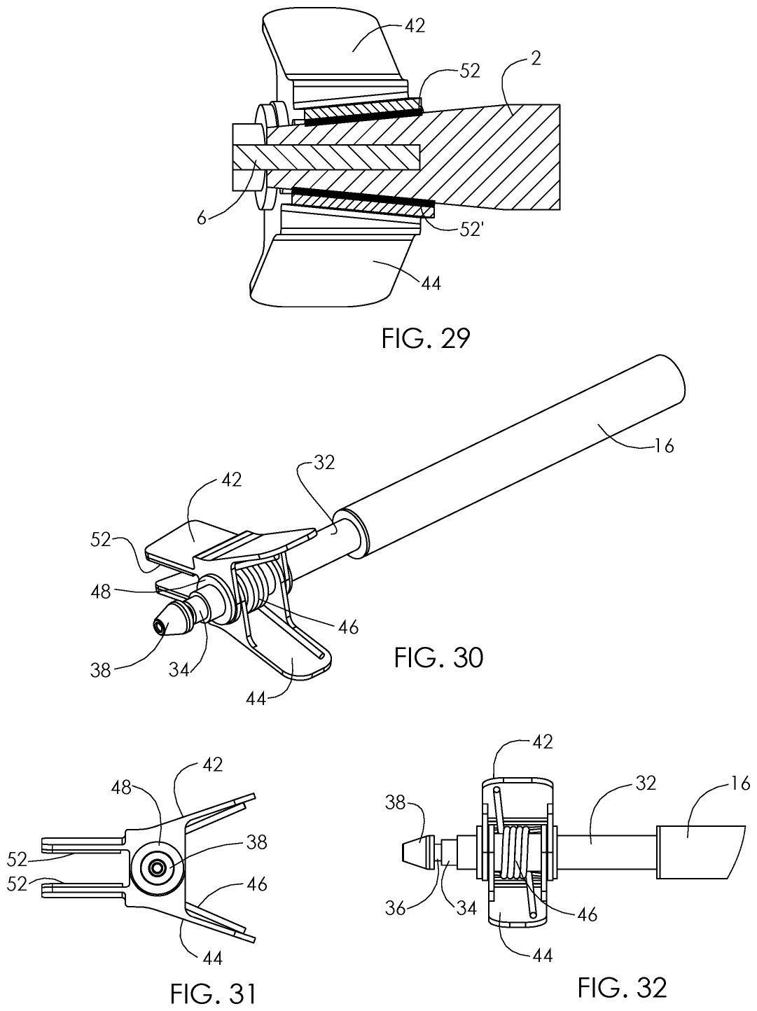

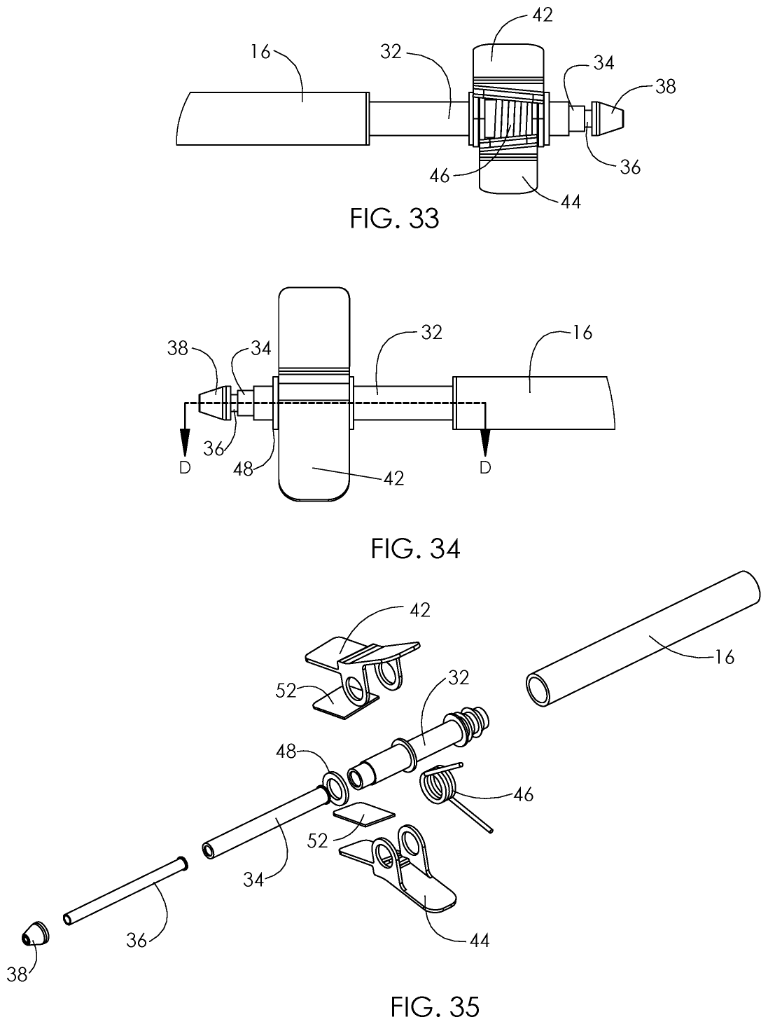

A spring biased clip assembly is operatively associated with the distal body portion 32 for detachably securing the smoke evacuation device 30 to the body 2 of surgical instrument 10 at a desirable location and angular orientation on either side of the body 2 with respect to the end effector 6, as illustrated in FIGS. 20 and 21, for example. The spring biased clip assembly includes upper and lower cooperating clip portions 42, 44 and a coiled torsion spring 46. Soft rubber plates 52 and 52′ are respectively associated with the interior clip/contact area 64 of each clip portion 42, 44 for securely gripping the body 2 of the electrosurgical instrument 10, as illustrated in FIG. 29. The clip portions 42, 44 each have circular opening 66 (see FIGS. 42 through 44) for supporting the clip assembly on the distal body portion 32 between a proximal flange 33 and a securing ring 48, as best seen in. FIGS. 33 and 34.

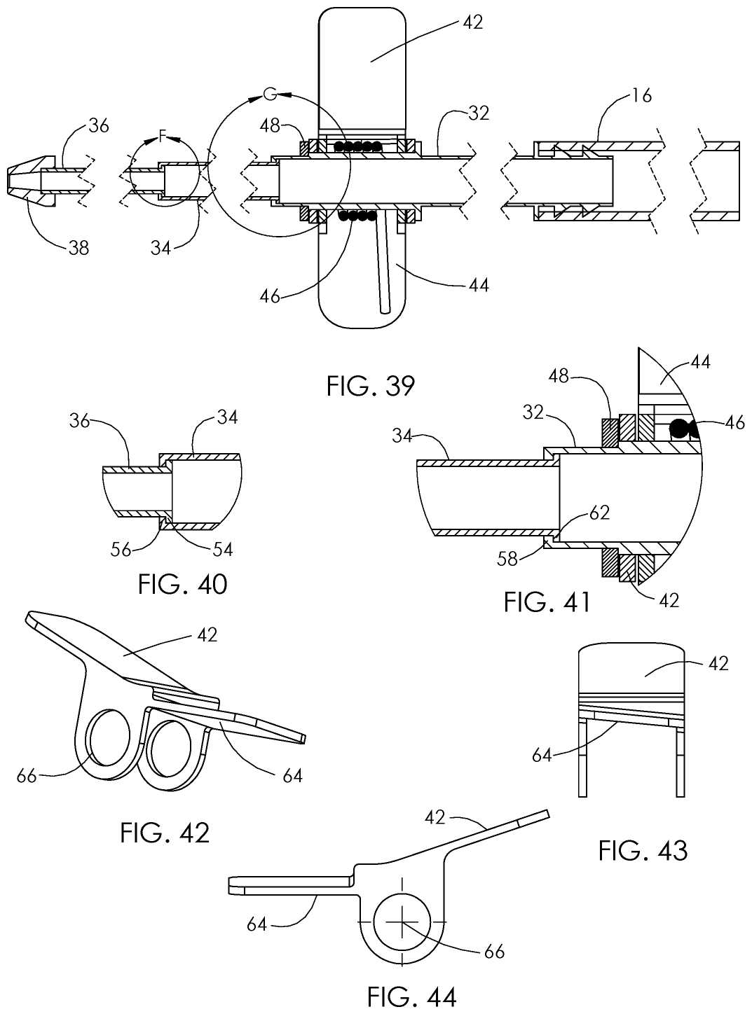

As shown in FIGS. 35 through 38, the distal body portion 32 of smoke evacuation device 30 has three telescopically nested tubular telescopic portions including a proximal portion 32, a medial portion 34 and a distal portion 36. A stop flange 54 is provided at the proximal end of tube 36 for interacting with a stop flange 56 at the distal end of the tube 34, and a stop flange 62 is provided at the proximal end of tube 34 for interacting with a stop flange 58 at the distal end of tube 32 to limit the extension of one telescoping tube relative to another, as illustrated in in FIGS. 39 through 41.

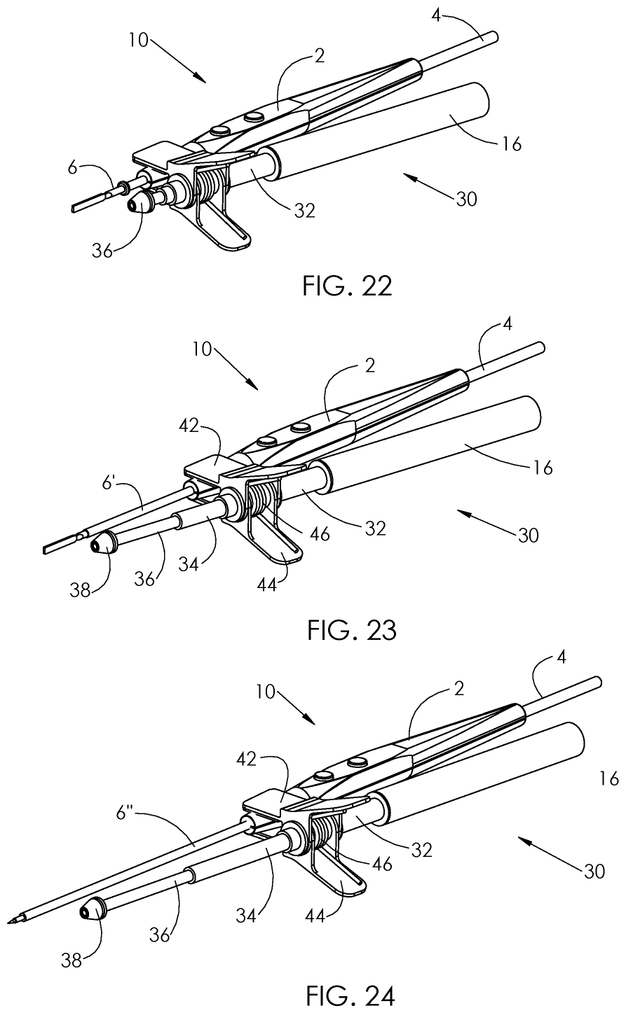

A suction tip 38 is provided at the distal end of the distal telescopic portion 36. A user can pull out a telescoping portion (or multiple telescoping portions) relative to the distal body portion 32 to extend the length of the smoke evacuation device 30. The proximal portion 16 remains fixed relative to the body 2 of instrument 10, while the nested, telescoping portions (32, 34 and 36) can be adjusted for optimal positioning to allow for improved suction at the site where smoke is generated while providing flexibility to avoid anatomy when in a tight surgical pocket, as well as the ability to match the length of the electrocautery blade/end effector (6, 6′ and 6″), as illustrated in FIGS. 22 through 24.

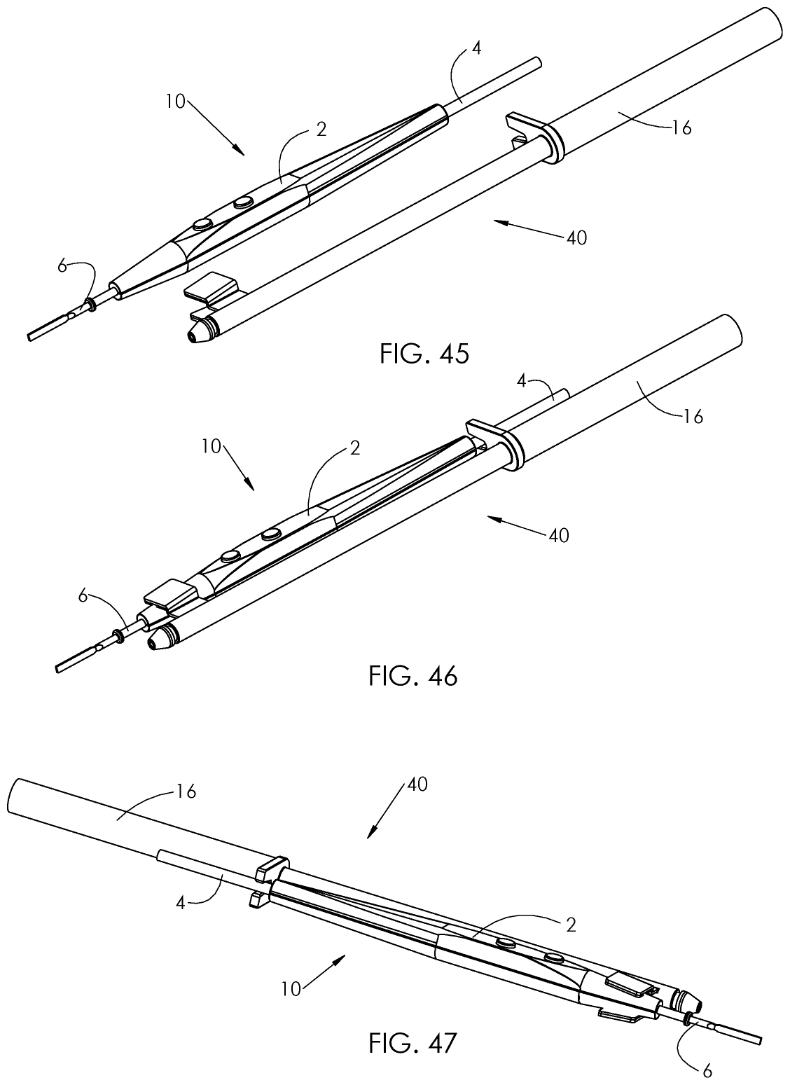

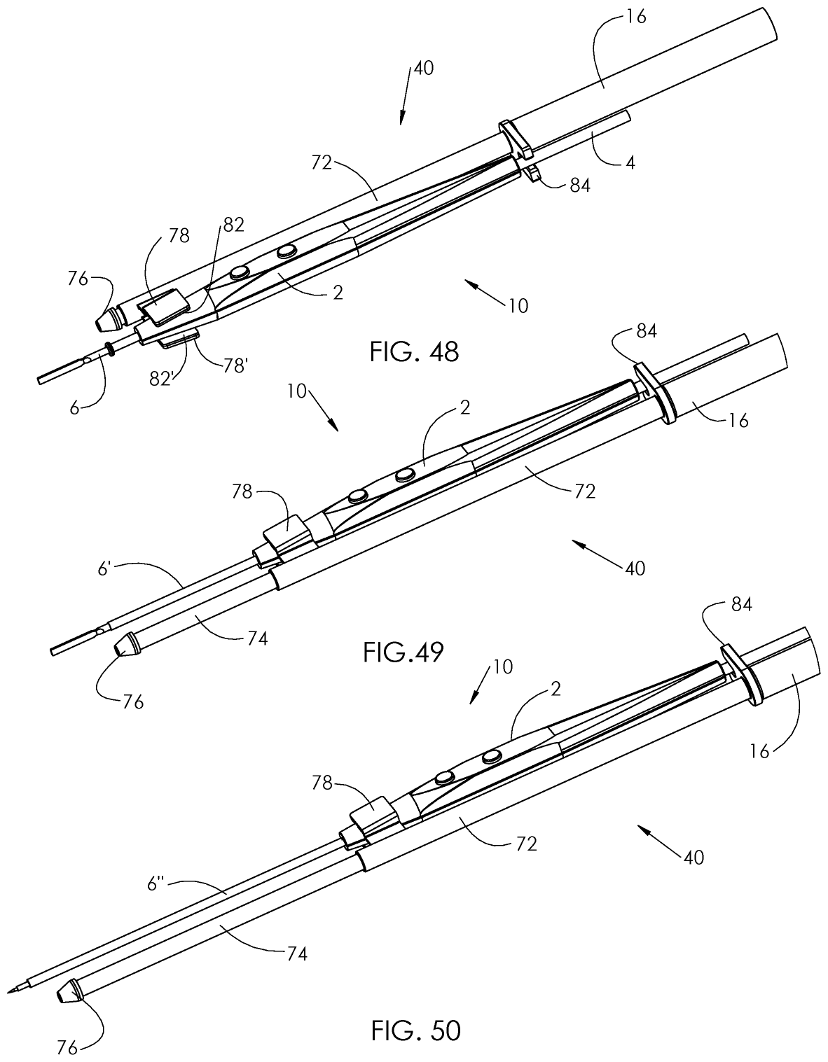

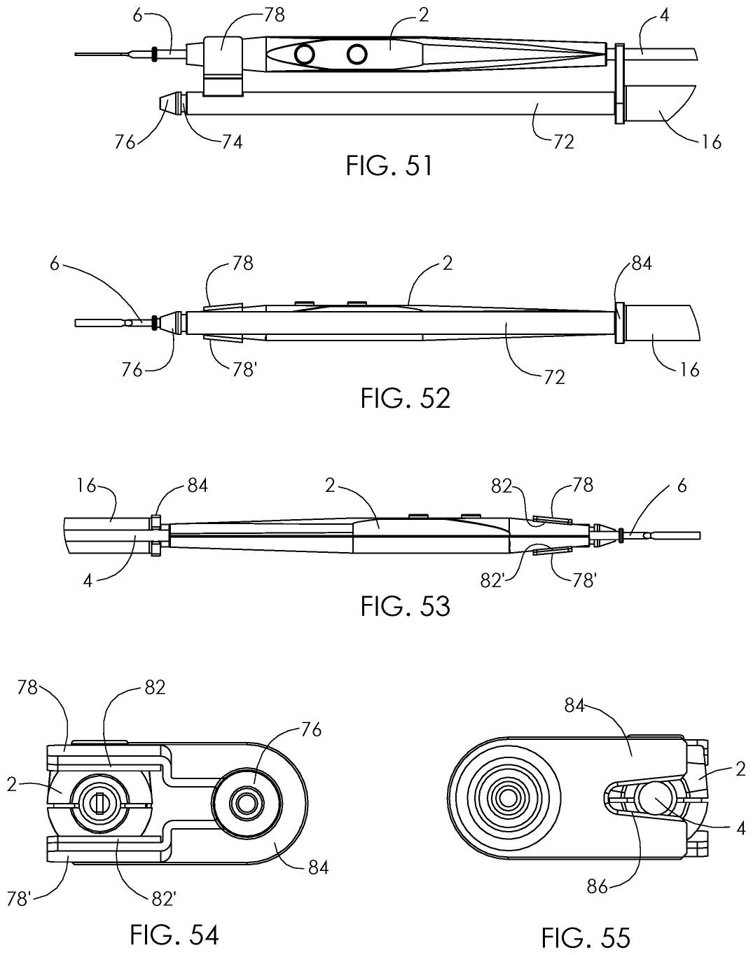

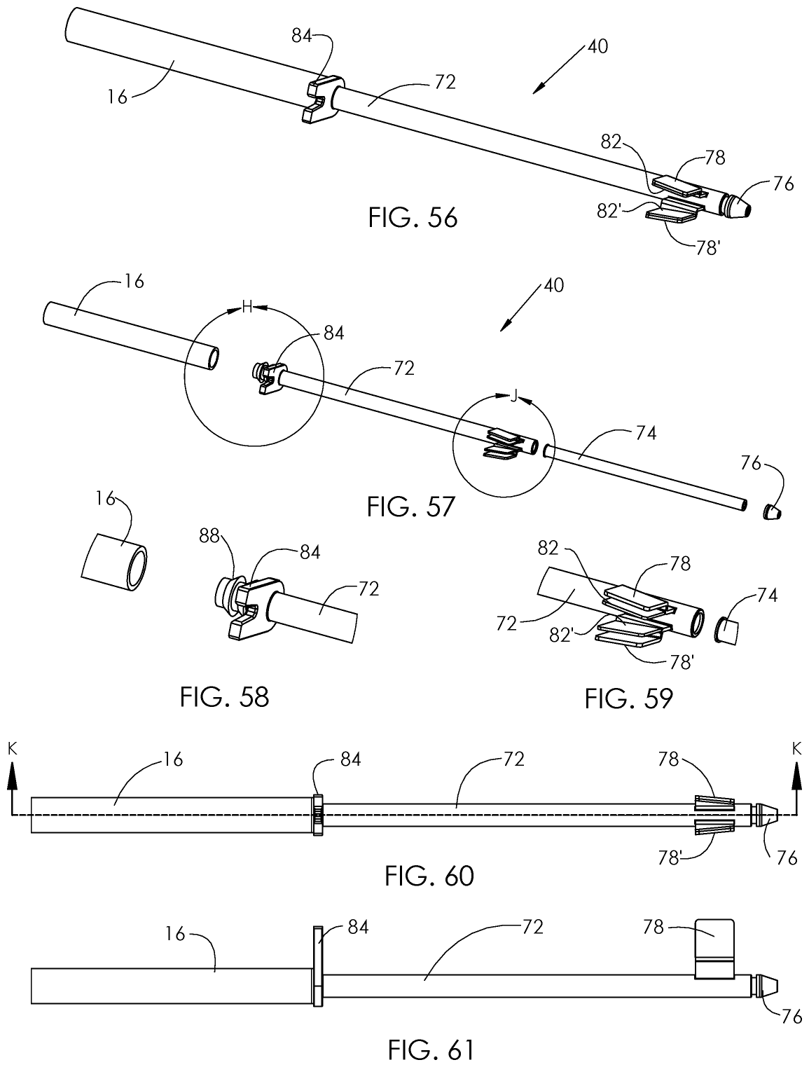

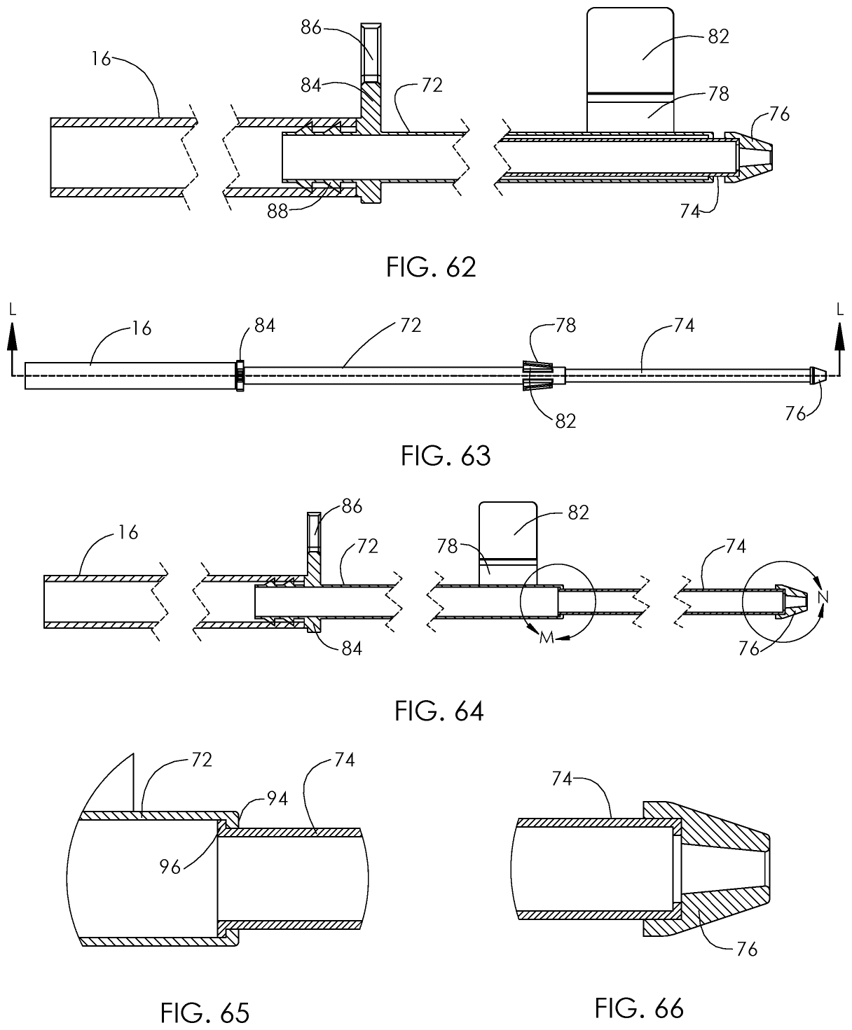

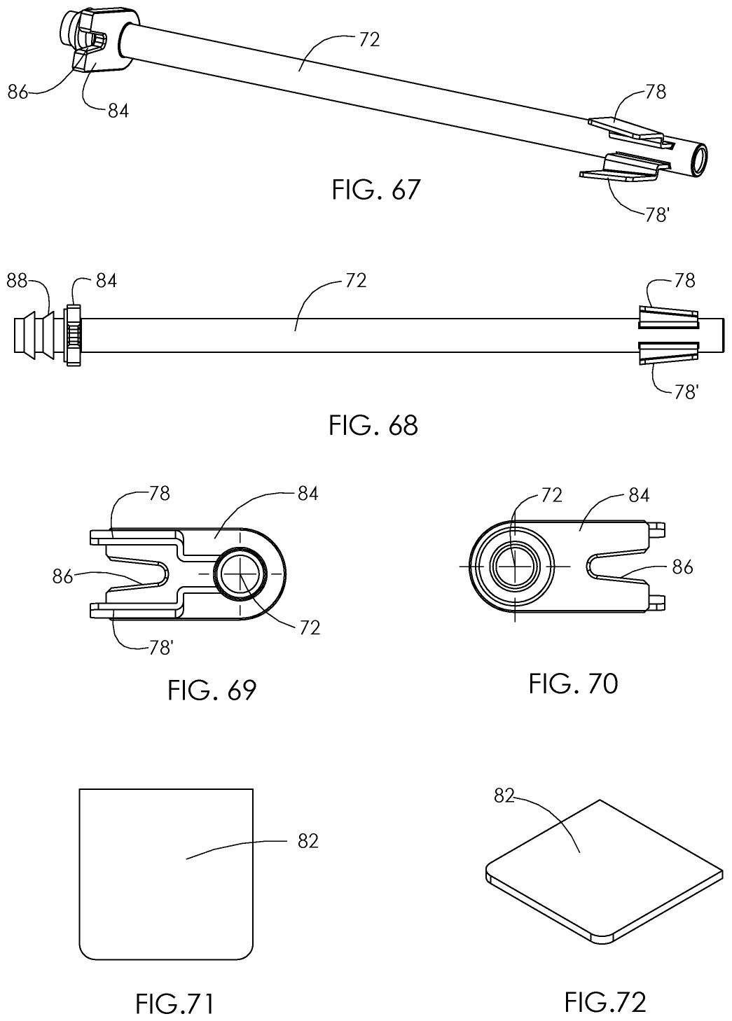

Referring now to FIGS. 45 through 72, there is illustrated another new and useful smoke evacuation device 40 for use with a handheld electrosurgical instrument 10 having an elongated body 2. In this embodiment of the invention, the smoke evacuation device 40 includes a proximal body portion 16, a distal body portion 72 and a telescoping portion 74 with a distal suction tip 76. A sealing barb 88 is provided at the proximal end of distal body portion 72 for engagement within the distal end of proximal body portion 16. A stop flange 96 is provided at the proximal end of tube 74 for interacting with a stop flange 94 at the distal end of the distal body portion 72, as best seen in FIGS. 64 and 65.

Smoke evacuation device 40 includes a distal attachment feature in the form of a bendable or flexible clipping structure having a pair of spaced apart deflectable arms 78 and 78′ with compliant (e.g., soft rubber) contact plates 82 and 82′, respectively, for securing the smoke evacuation device 40 to the distal end portion of the body 2 of surgical instrument 10 (see FIG. 54). In addition, the smoke evacuation device 40 incudes a proximal attachment feature in the form of a relatively rigid cable locking clip 84 with an interior profile portion 86 that is adapted and configured to snap onto the soft insulation of the cable 4 extending from the proximal end of the body 2 of surgical instrument 10 (see FIG. 55).

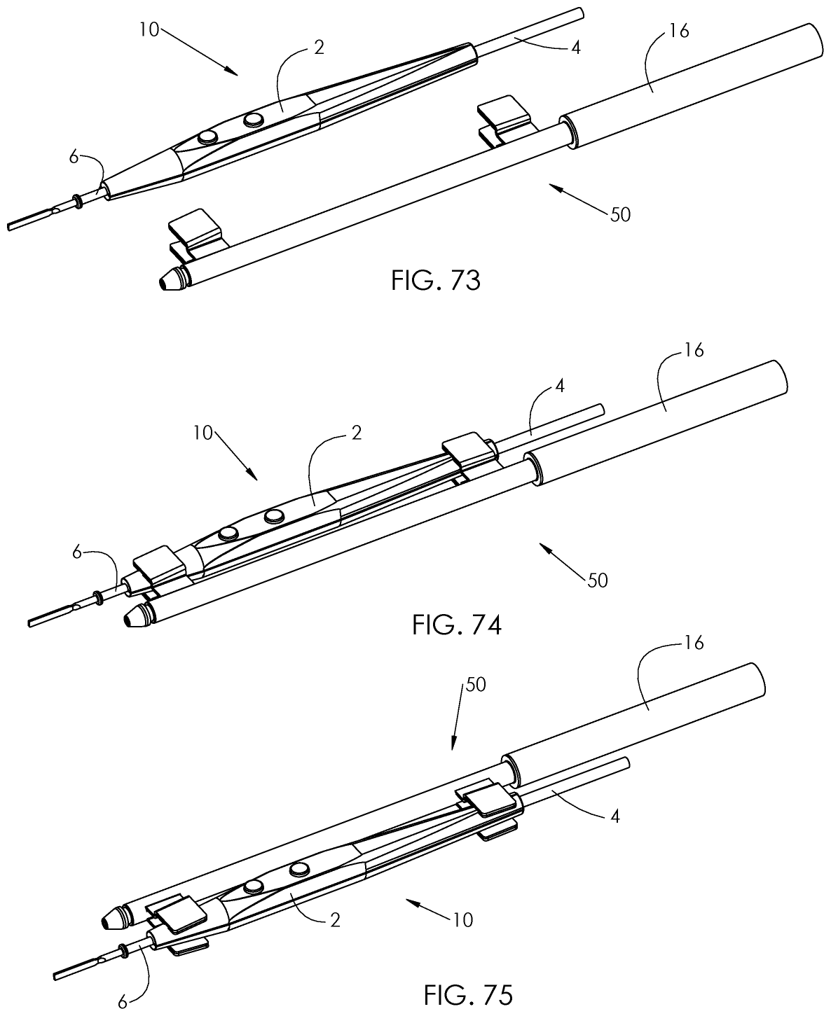

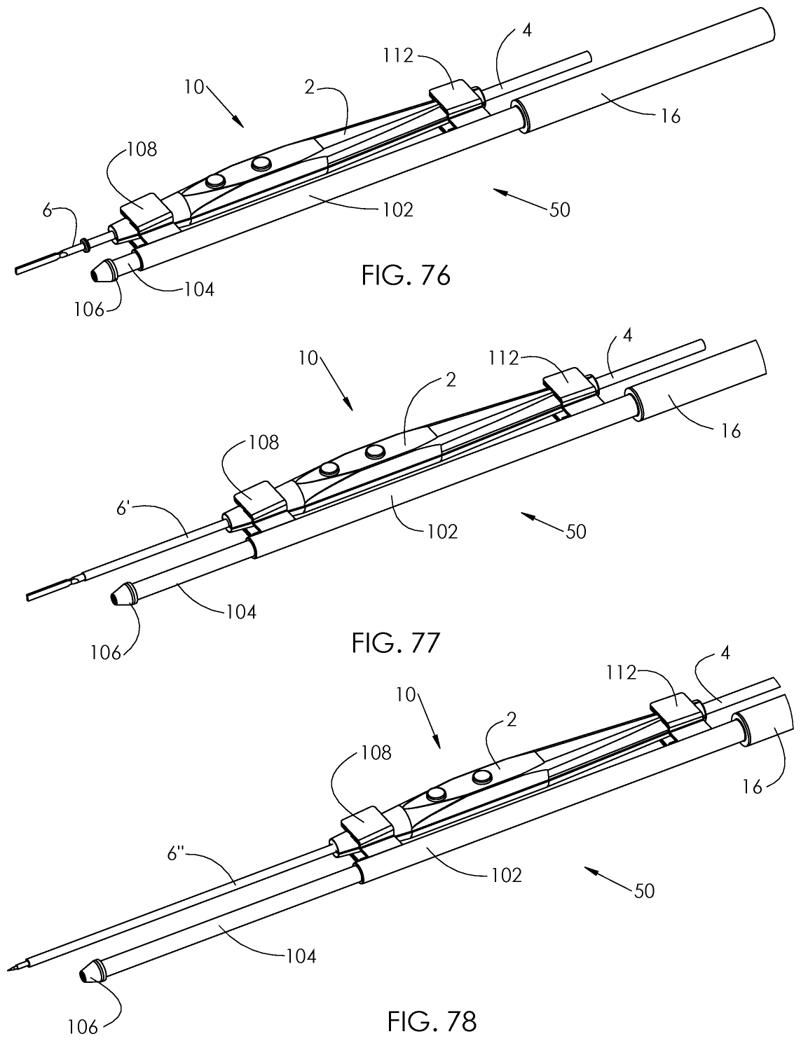

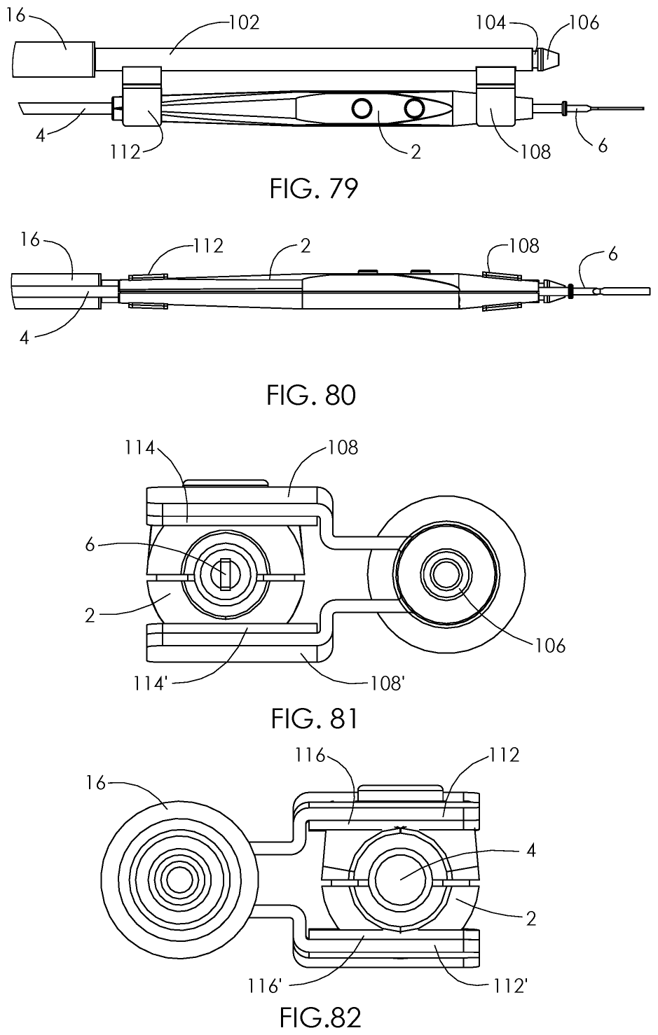

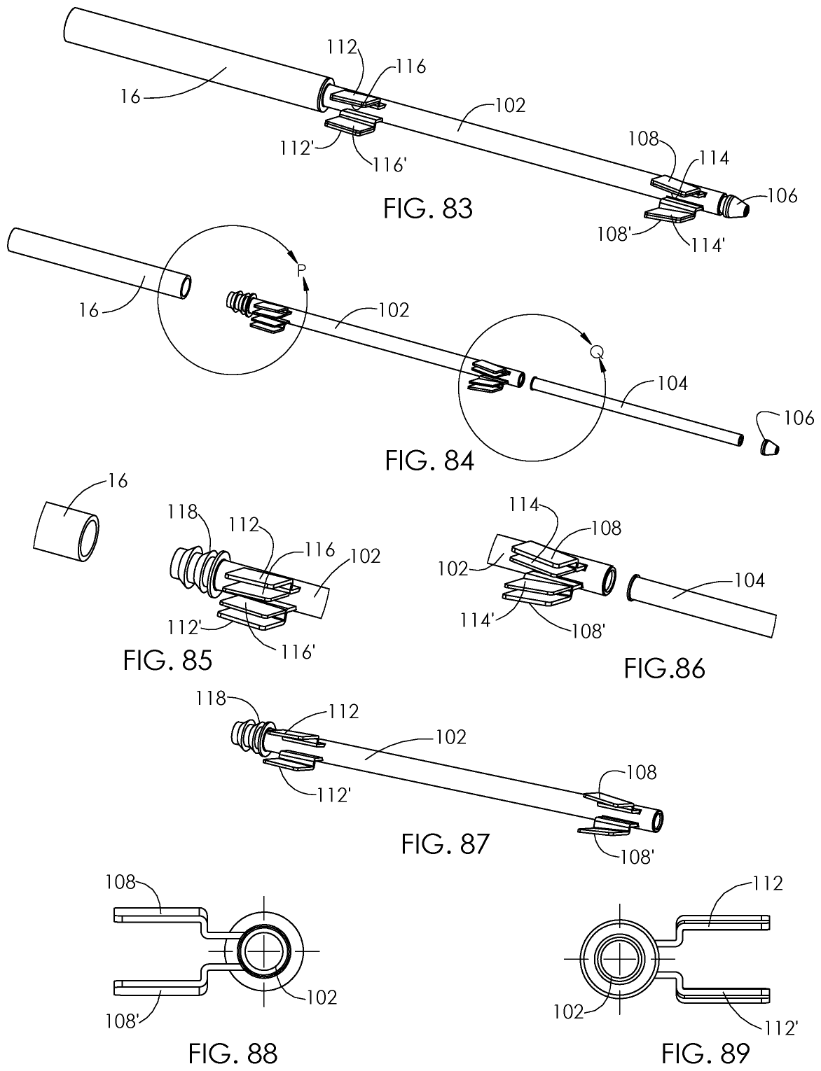

Referring now to FIGS. 73 through 89, there is illustrated another new and useful smoke evacuation device 50 for use with a handheld electrosurgical instrument 10 having an elongated body 2. In this embodiment of the invention, the smoke evacuation device 50 includes a proximal body portion 16, a distal body portion 102 and a telescoping portion 104 with a distal suction tip 106. A sealing barb 118 is provided at the proximal end of distal body portion 102 for engagement within the distal end of proximal body portion 16, as shown in FIG. 85. A stop flange is provided at the proximal end of tube 104 for interacting with a stop flange at the distal end of the distal body portion 102, as shown in FIG. 86.

Smoke evacuation device 50 further includes a distal attachment feature in the form of a bendable clipping structure including a pair of spaced apart deflectable arms 108 and 108′ with compliant (e.g. soft rubber) contact plates 114 and 114′, respectively, for securing the device to the distal end of the body 2 of surgical instrument 10, as shown in FIGS. 81 and 88. In addition, the smoke evacuation device 50 incudes a proximal attachment feature also in the form of a bendable clipping structure including a pair of spaced apart deflectable arms 112 and 112′ with compliant (e.g. soft rubber) contact plates 116 and 116′, respectively, for securing the device to the proximal end of the body 2 of surgical instrument 10, as shown in FIGS. 82 and 89.

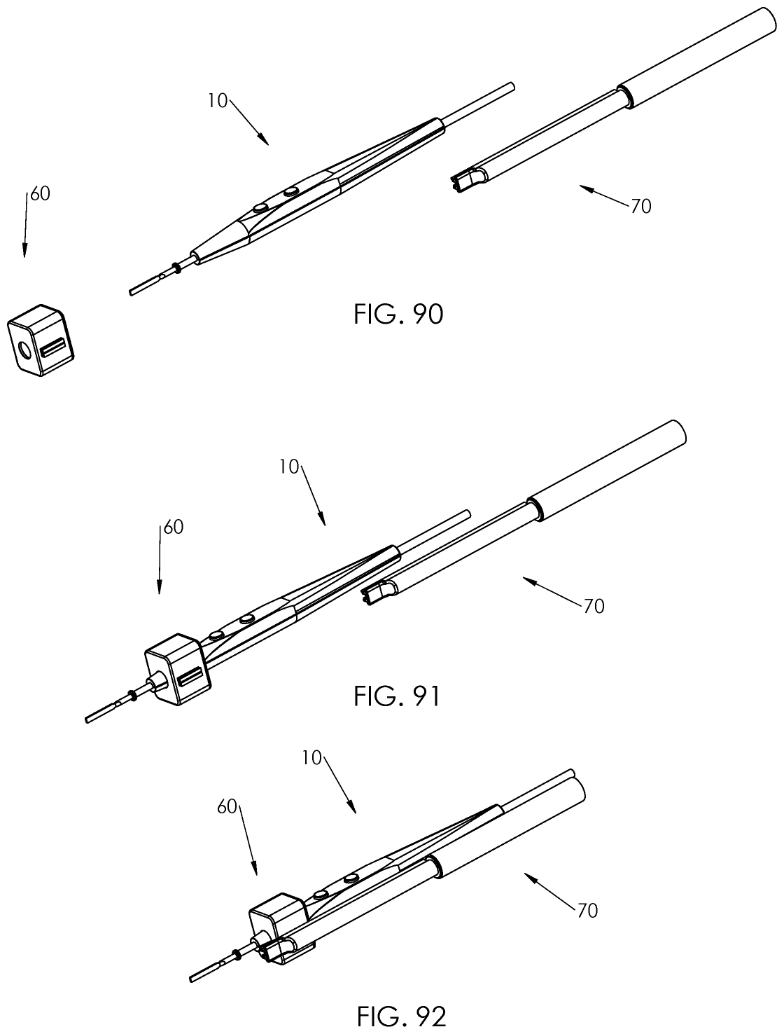

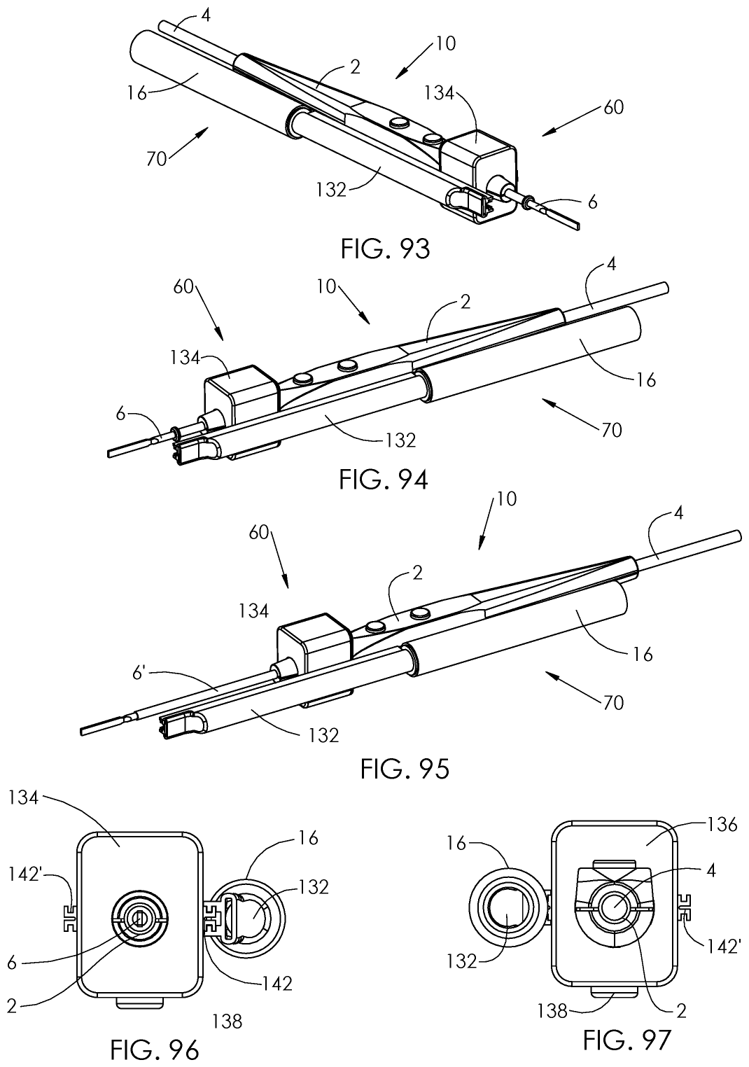

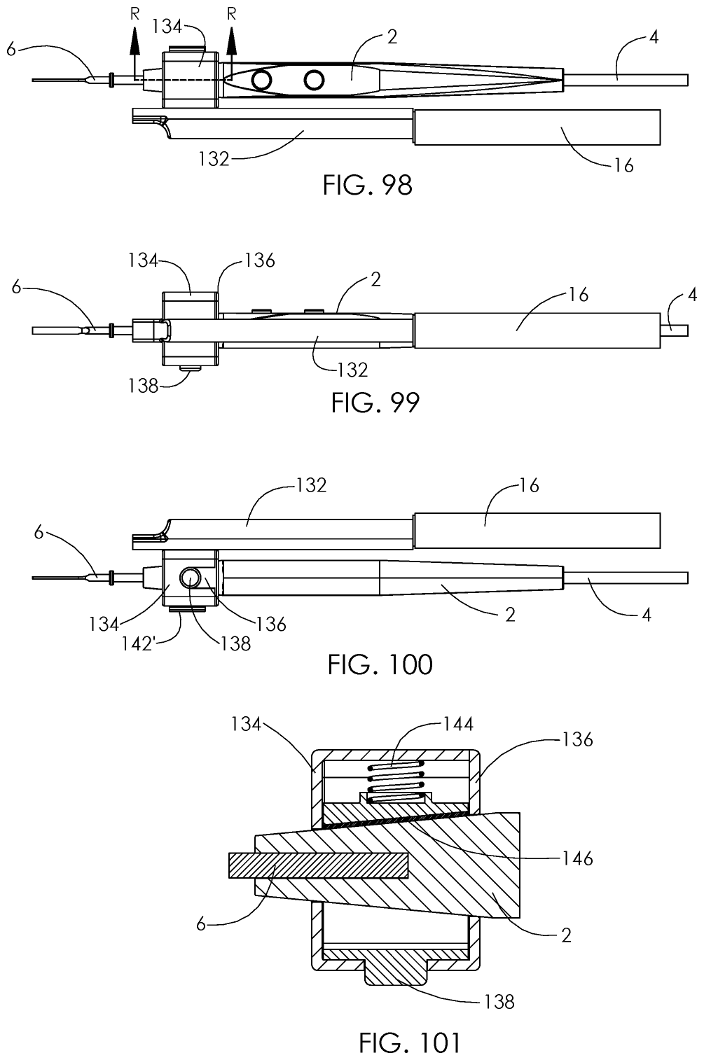

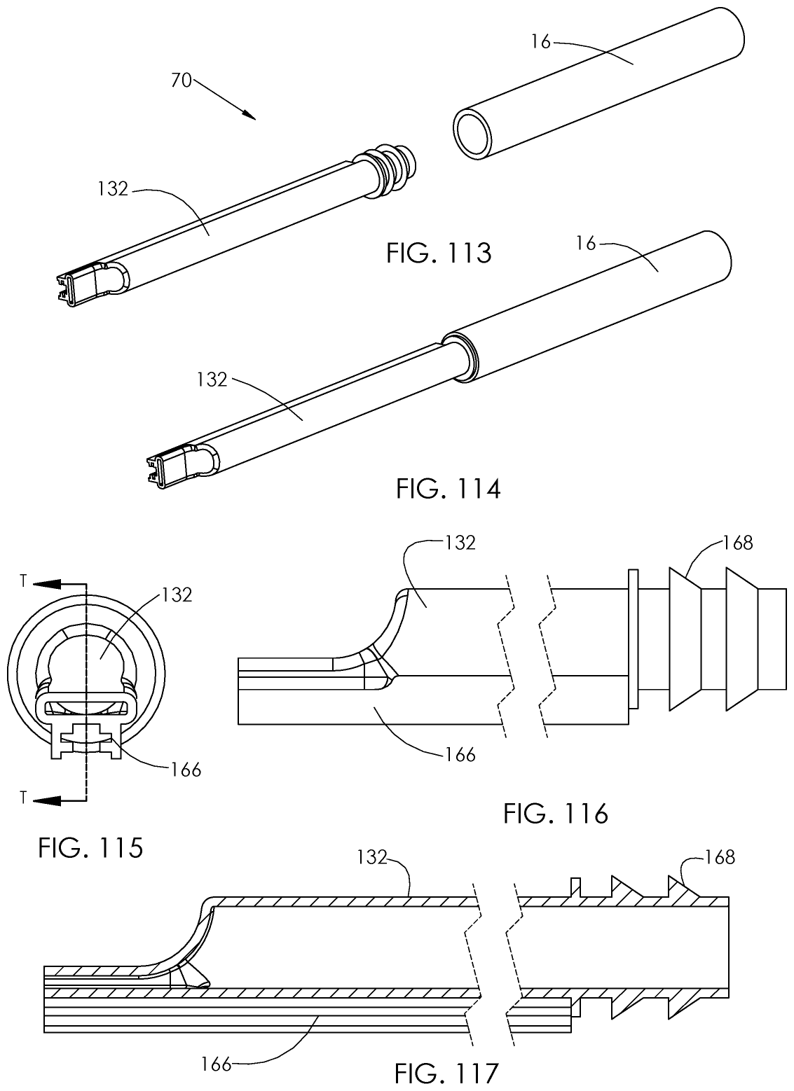

Referring now to FIGS. 90 through 117, there is illustrated another new and useful smoke evacuation device 70 for use with a handheld electrosurgical instrument 10 having an elongated body 2. In this embodiment of the invention, the smoke evacuation device 70 includes a proximal body portion 16 and a distal body portion 132 with a distal suction tip. A sealing barb 168 is provided at the proximal end of distal body portion 122 for engagement within the distal end of proximal body portion 16, as shown in FIG. 116.

In this embodiment of the subject invention, the distal attachment feature is in the form of a mounting block 60 that includes a rectangular housing 134 having a rear cover 136. A rectangular engagement ring 138 resides within the housing 134 and it includes a compliant interior plate 146 for compressively engaging the distal end portion of the body 2 of surgical instrument 10, as best seen in FIG. 101. The engagement ring 138 is biased by a coiled compression spring 144 that is seated in a recess 156 on the top surface thereof.

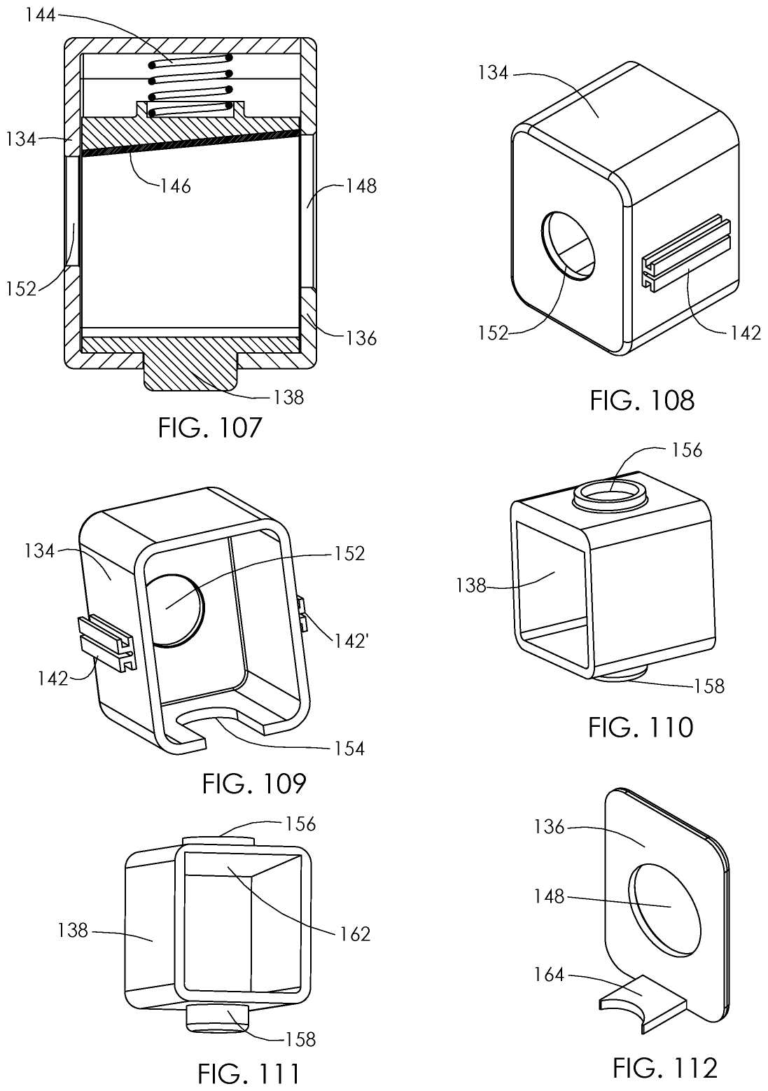

A passage extends through the housing 134 for accommodating the distal end portion of the body 2 of surgical instrument 10. The passage is defined by a port 148 in rear cover 136 and a corresponding aligned port 152 in the front wall of the housing 134. As best seen in FIG. 107, a push button 158 is formed on the bottom surface of the engagement ring 138 for moving the engagement ring against the bias of coiled spring 144 to release the surgical instrument 10 therefrom. The push button 158 extends through an aperture 154 formed in the bottom surface of the housing 134 and defined in part by a protrusion 164 on the rear cover 136 (see FIG. 112).

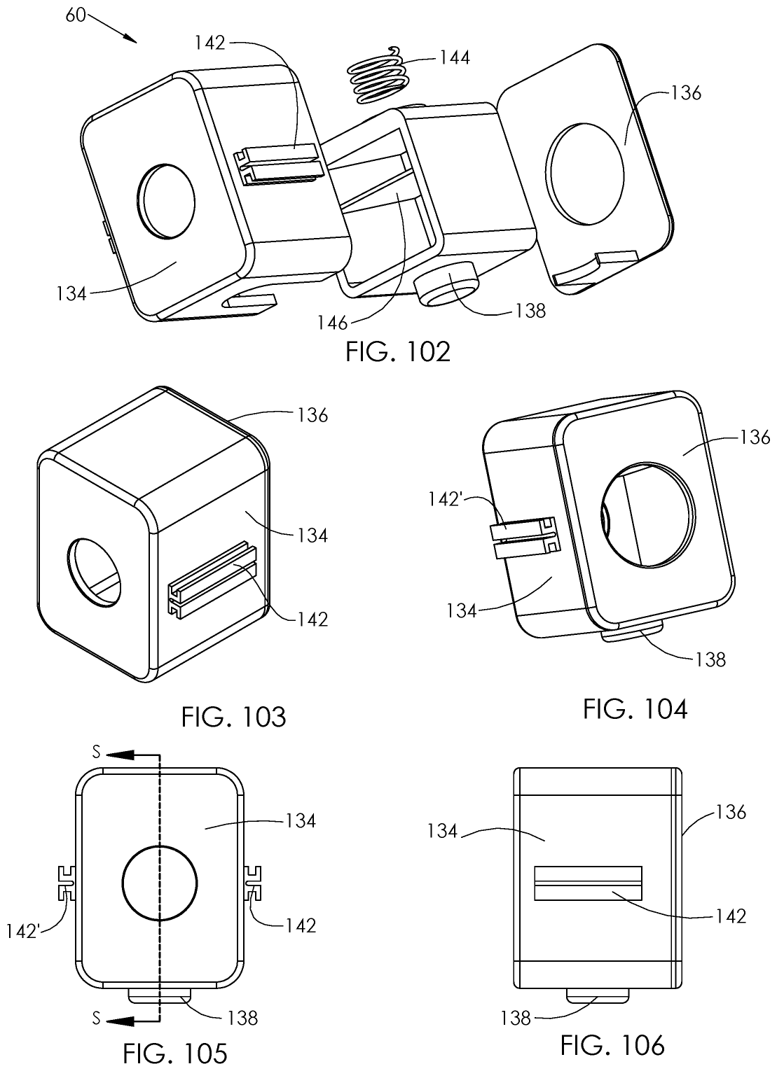

As best seen in FIGS. 103 through 106, a pair of rails 142 and 142′ are provided on the opposed side walls of the housing 134. The rails 142 and 142′ are adapted and configured for selective sliding cooperative engagement with an elongated track 166 that extends along the length of the distal body portion 132, as best seen in FIGS. 115 through 117. In use, regardless of the side of the mounting block 60 the smoke evacuation device 70 is supported upon, it can be slidingly extended and retracted relative to the mounting block 60 along the track 166 for optimal positioning to allow for improved suction at the site where smoke is generated while providing flexibility to avoid anatomy when in a tight surgical pocket, as well as the ability to match the length of the electrocautery blade/end effector (6 and 6′), as illustrated for example in FIGS. 94 and 95.

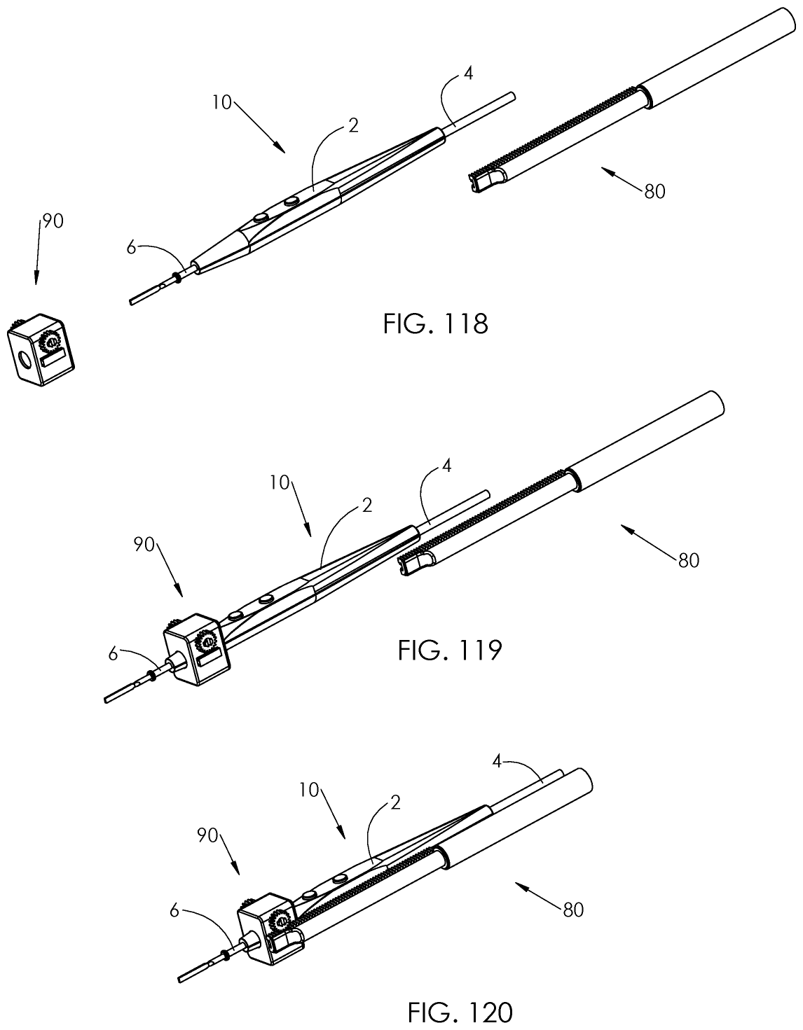

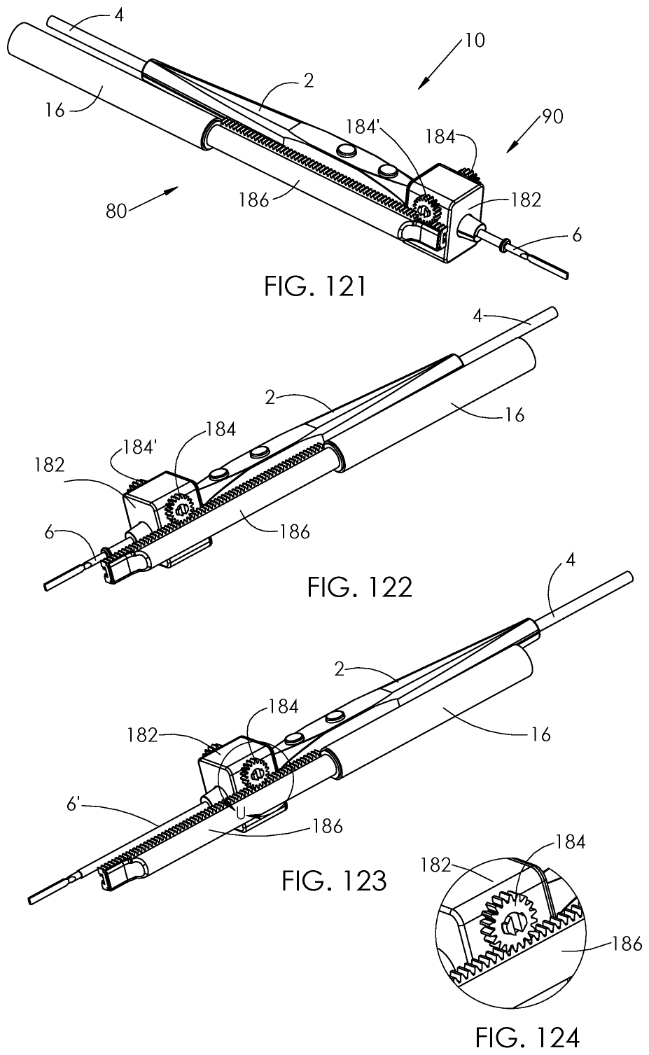

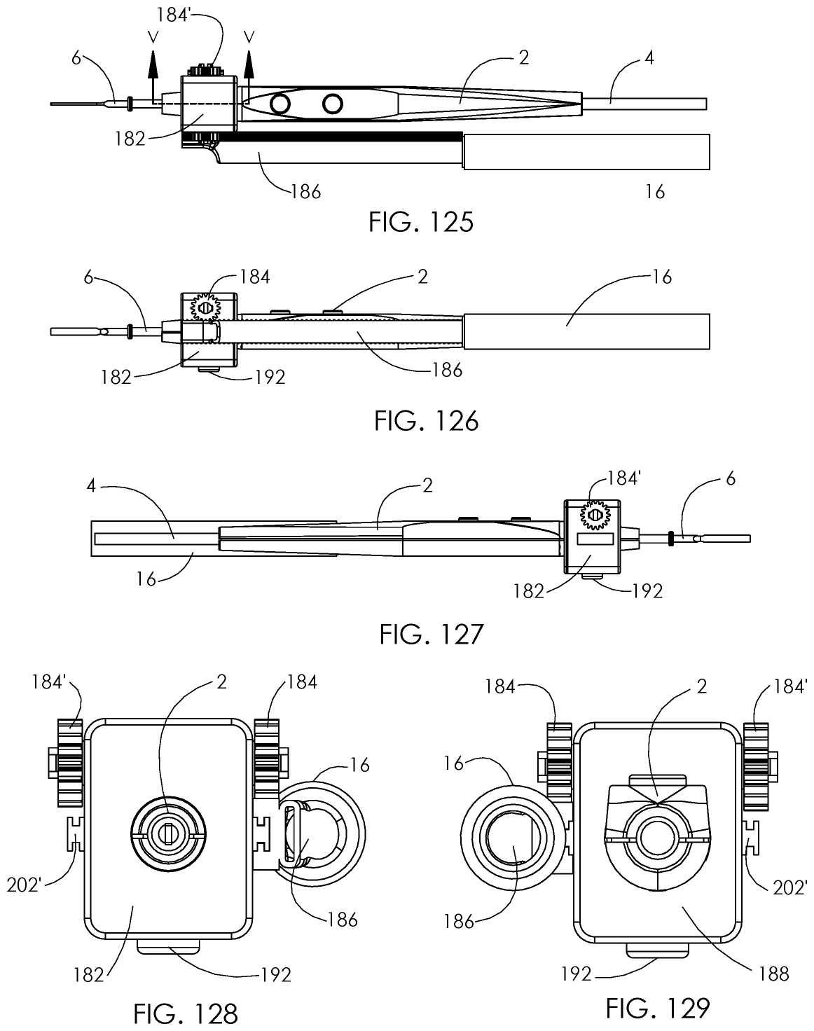

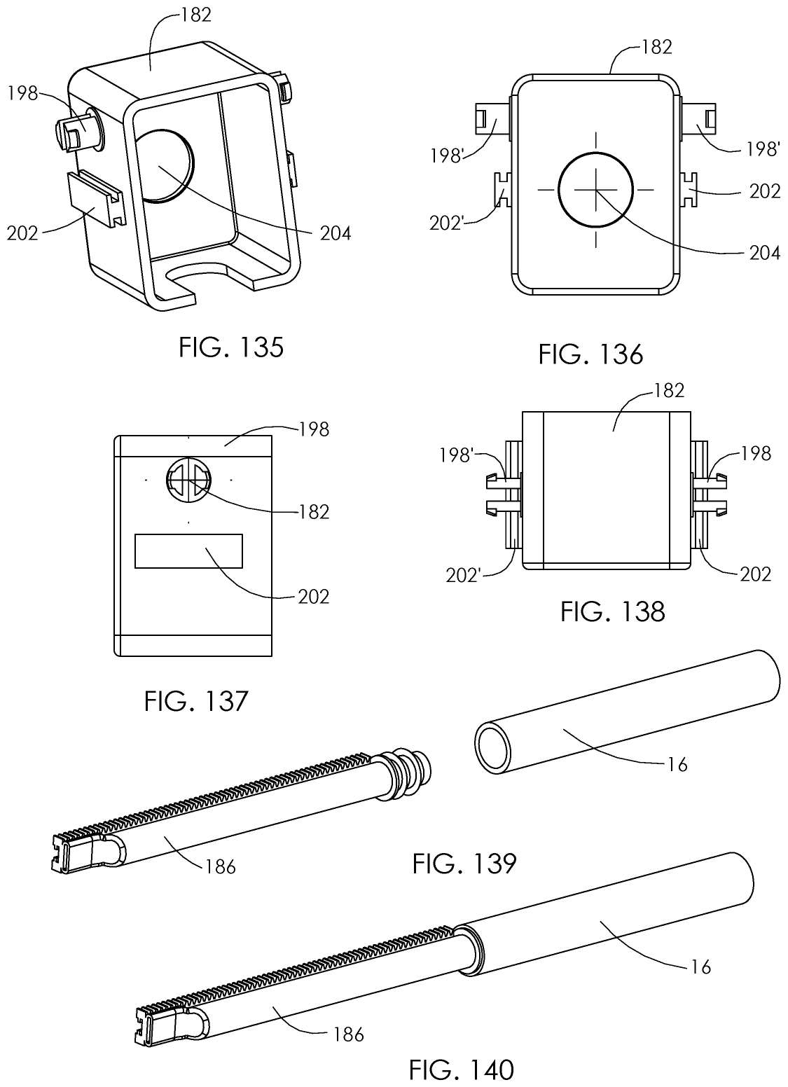

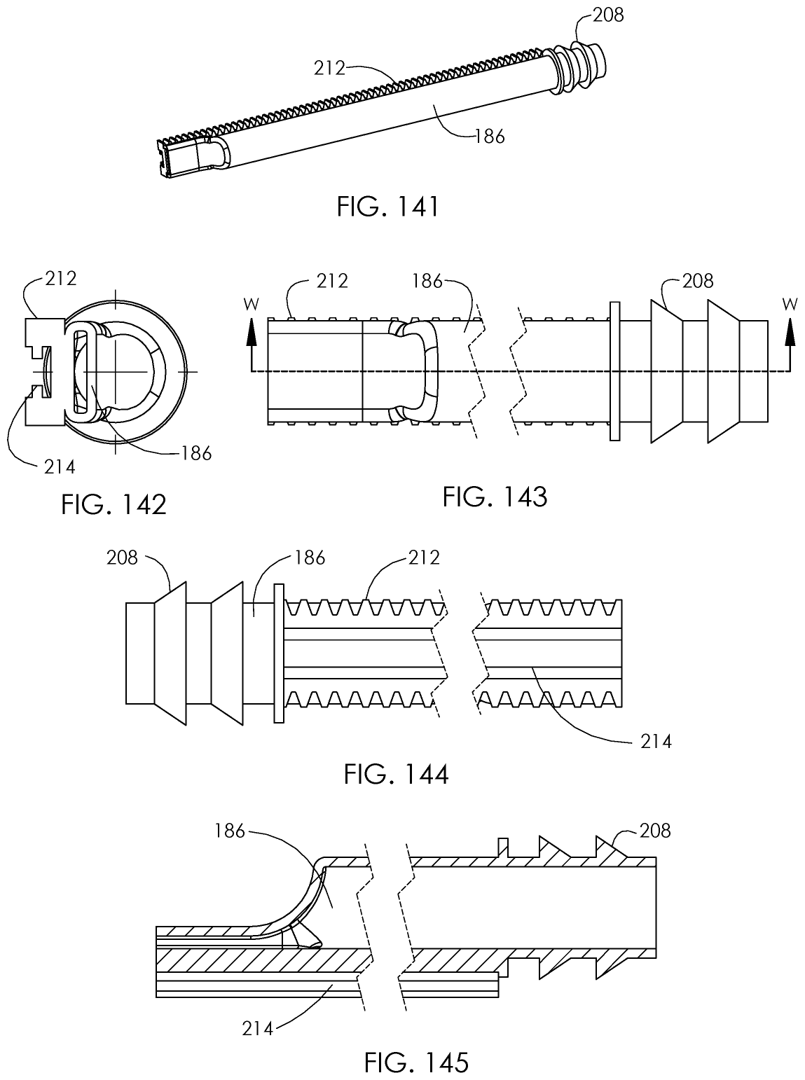

Referring now to FIGS. 118 through 145, there is illustrated another new and useful smoke evacuation device 80 for use with a handheld electrosurgical instrument 10 having an elongated body 2. In this embodiment of the invention, the smoke evacuation device 80 includes a proximal body portion 16 and a distal body portion 186 with a distal suction tip. A sealing barb 208 is provided at the proximal end of distal body portion 186 for engagement within the distal end of proximal body portion 16 (see FIG. 141).

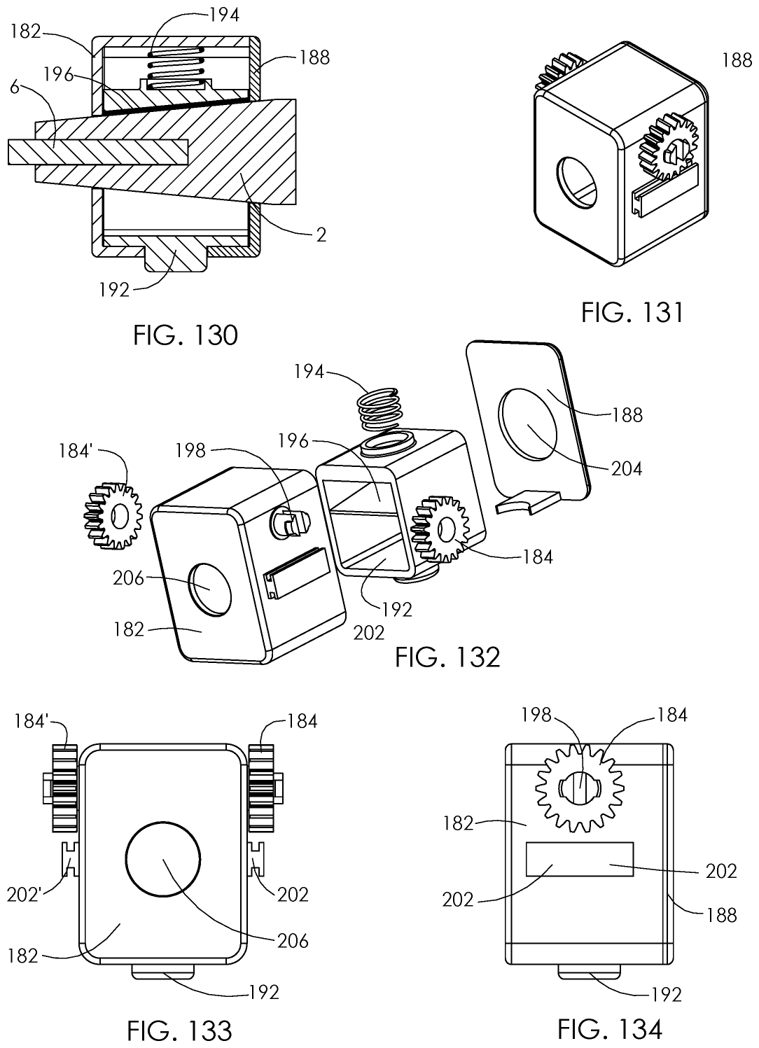

In this embodiment of the subject invention, the distal attachment feature is once again in the form of a mounting block 90 that included a rectangular housing 182 having a rear cover 188. A rectangular engagement ring 192 resides within the housing 182 and it includes a compliant interior plate 196 for compressively engaging the distal end portion of the body 2 of surgical instrument 10, as best seen in FIG. 130. The engagement ring 192 is biased by a coiled compression spring 194 that is seated in a recess on the top surface thereof.

A passage extends through the housing 182 for accommodating the distal end portion of the body 2 of surgical instrument 10. The passage is defined by a port 204 in rear cover 188 and a corresponding aligned port 206 in the front wall of the housing 182. A push button 192 is formed on the bottom surface of the engagement ring 192 for moving the engagement ring against the bias of coiled spring 194. The push button 158 for releasing the surgical instrument 10 extends through an aperture formed in the bottom surface of the housing 182 and defined in part by a protrusion on the rear cover 188 (see FIG. 132).

As best seen in FIGS. 133 through 138, a pair of rails 202 and 202′ are provided on the opposed side walls of the housing 182 for selective sliding cooperative engagement with an elongated track 214 that extends along the length of the distal body portion 186, as best seen in FIGS. 142 through 145. In this embodiment of the invention, pinion gears 184 and 184′ are mounted on respective side axles 198 that extend from the opposed sidewalls of housing 182. The pinion gears 184 and 184′ cooperate with an elongated gear rack 212 that is formed along the length of the distal body portion 186, as best seen in FIG. 124.

In use, regardless of the side of the mounting block 90 the smoke evacuation device 80 is supported upon, the smoke evacuation device 80 can be extended and retracted relative to the mounting block 90 along the track 166 through rotation of a pinion gear 184, 184′ for optimal positioning to allow for improved suction at the site where smoke is generated while providing flexibility to avoid anatomy when in a tight surgical pocket, as well as the ability to match the length of the electrocautery blade/end effector (6 and 6′), as illustrated in FIGS. 122 and 123. At such a time, friction between the rails 202, 202′ and the track 214 prevent accidental displacement of the distal body portion 186.

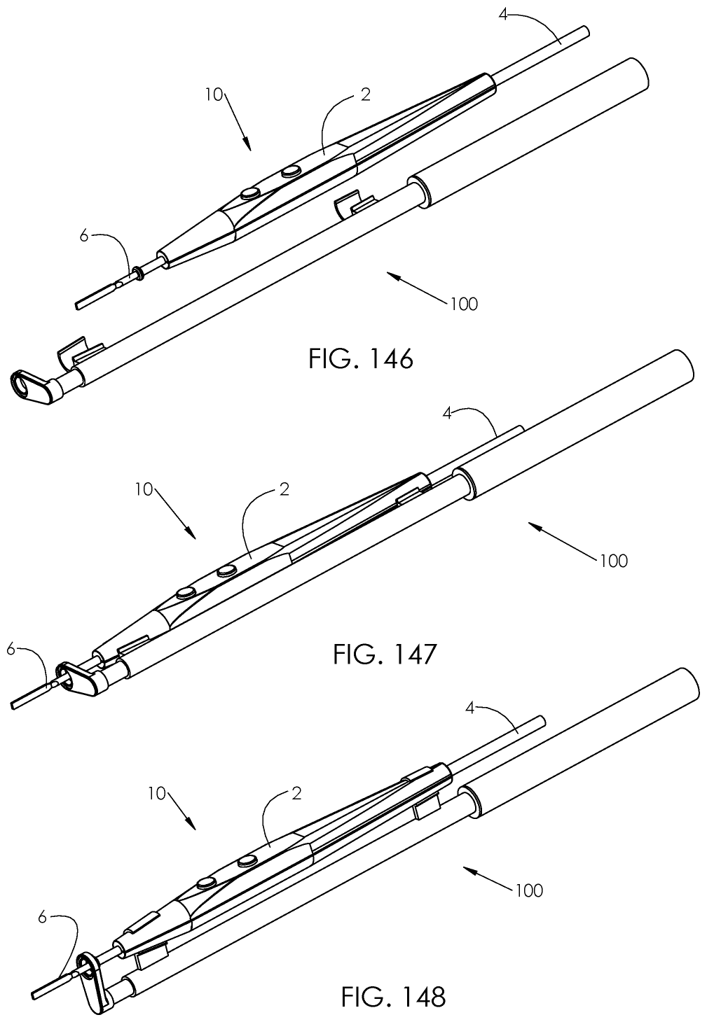

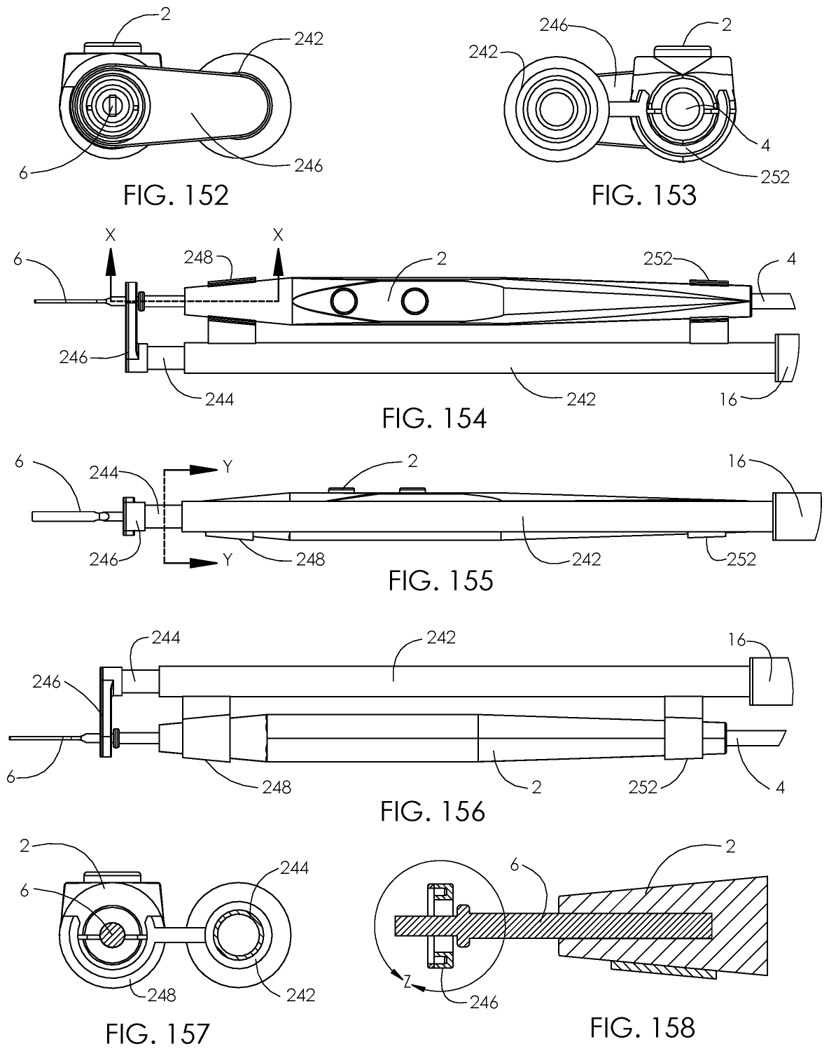

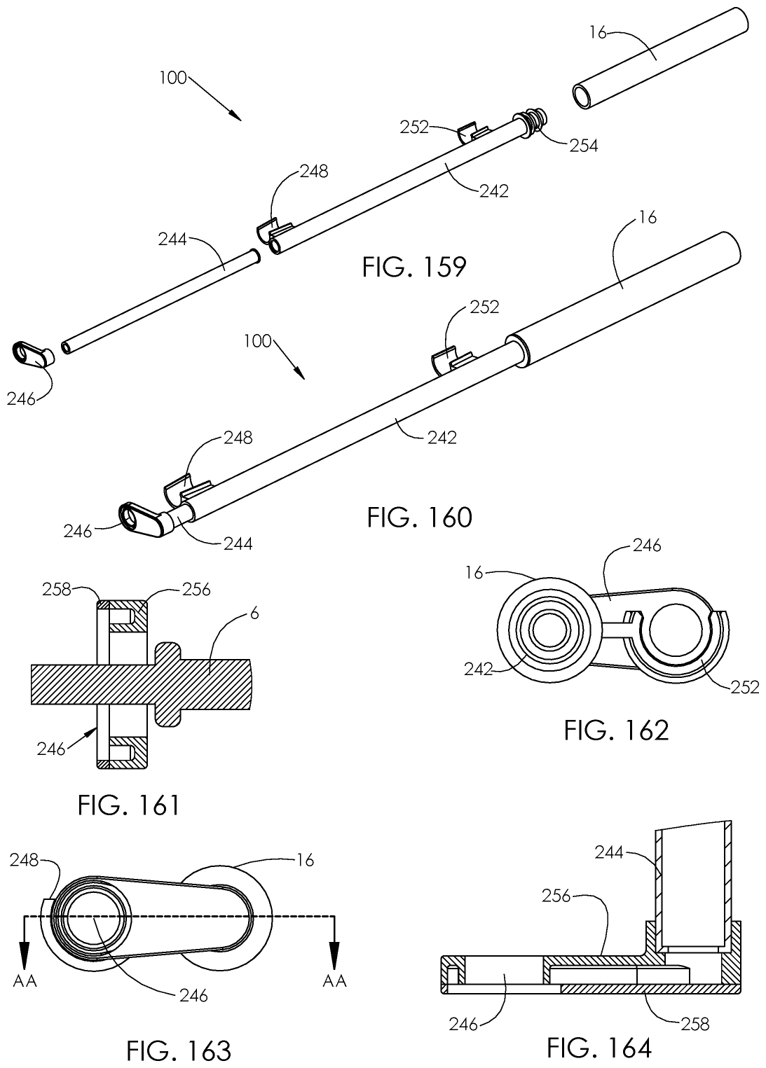

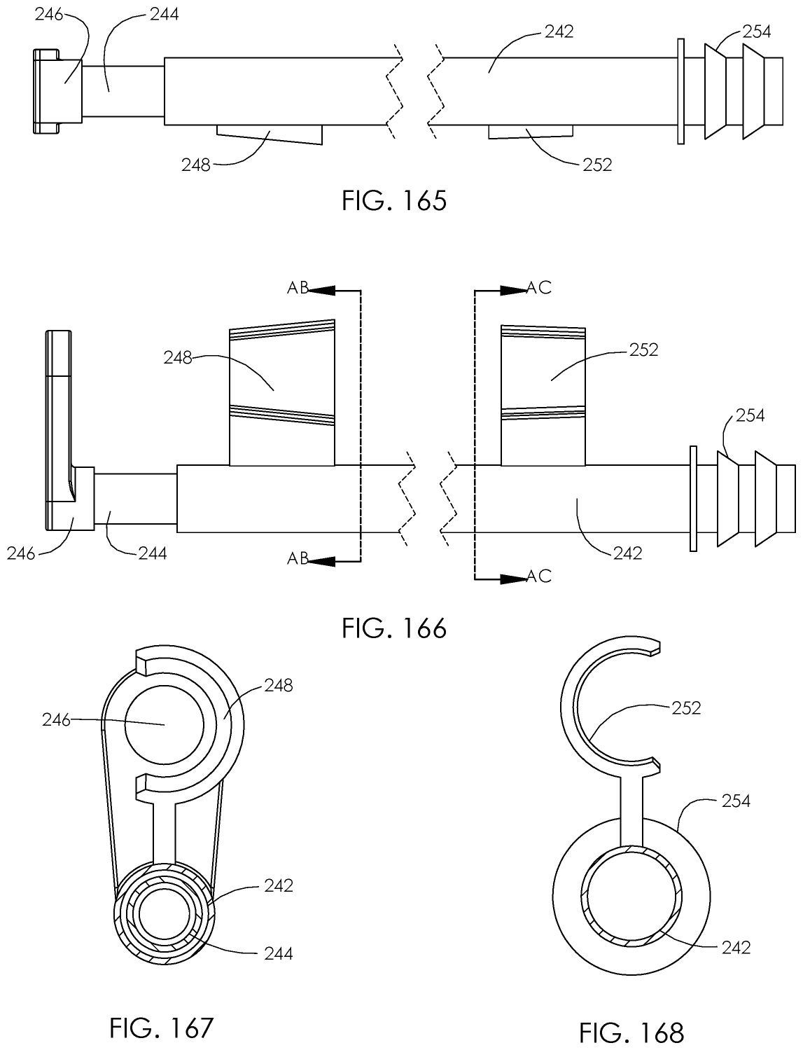

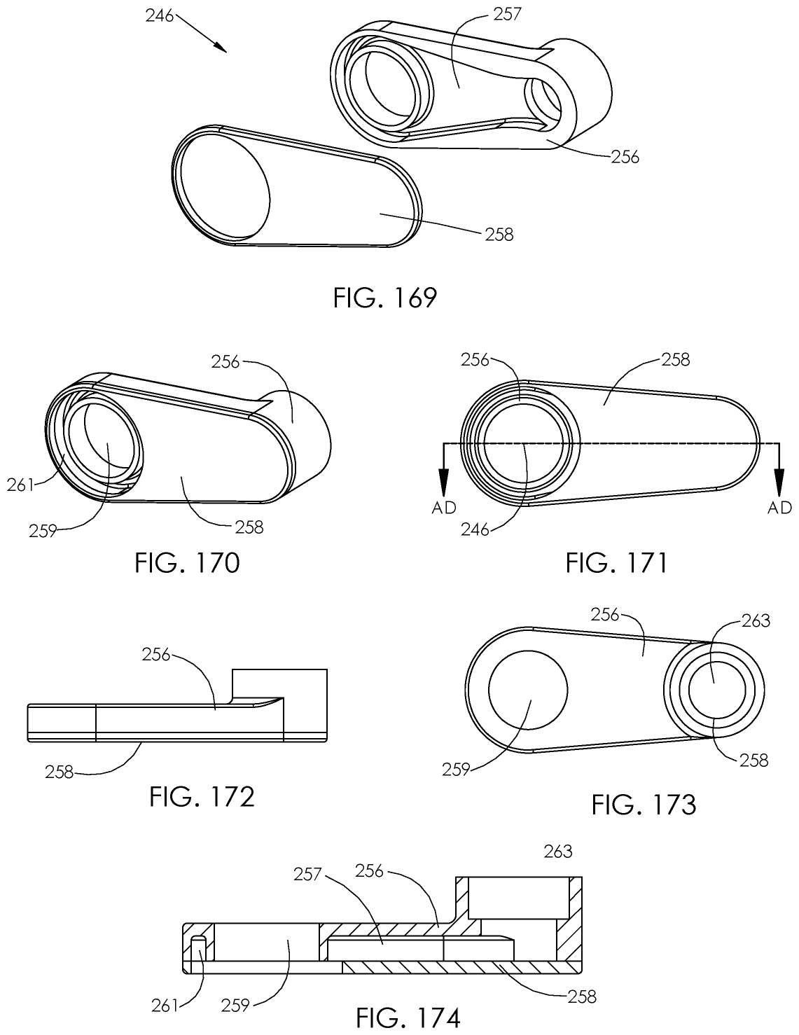

Referring to FIGS. 146 through 174, there is illustrated another new and useful smoke evacuation device 100 for use with a handheld electrosurgical instrument 10 having an elongated body 2. In this embodiment of the invention, the smoke evacuation device 100 includes a proximal body portion 16, a distal body portion 242 and a telescoping portion 244 with a distal suction tip assembly 246, best seen in FIGS. 163 through 164. Suction tip assembly 246 will be discuss in greater detail below with reference to FIGS. 169 through 174. A sealing barb 254 is provide at the proximal end of distal body portion 242 for engagement within the distal end of proximal body portion 16, and stop flanges are provided at the proximal end of telescoping portion 244 and the distal end of distal body portion 242, as best seen in FIG. 165.

Smoke evacuation device 100 includes a distal attachment feature in the form of a “C” shaped clamp 248 located adjacent the distal end of distal body portion 242 for clamping on to the proximal end of the body 2 of surgical instrument 10 and a proximal attachment feature in the form of another “C” shaped clamp 252 located adjacent the proximal end of distal body portion 242 for clamping on to the proximal end of the body 2 of surgical instrument 10, as best seen in FIGS. 165 through 168. The “C” shaped clamps 248 and 252 are flexible enough to allow the user to snap the smoke evacuation device 100 into place. Moreover, the clamps 248 and 252 are adapted and configured to enable the smoke evacuation device 100 to be secured to the left or right side of the body 2 of surgical instrument 10, or even to the underside of the surgical instrument 10 as shown in FIG. 148.

Turning now to FIGS. 169 through 174, the suction tip assembly 246 of smoke evacuation device 100 includes a tip body 256 defining an interior chamber 257 and a tip cover 258 for enclosing the chamber 257. An aperture 259 is provided at one end of tip assembly 246 for accommodating and surrounding the end effector 6. Aperture 259 also defines an annular inlet 261 to interior chamber 257. The body 256 further defines an outlet 263 of the interior chamber 257, which communicates with the distal end of the telescoping portion 244. Thus, smoke drawn into the inlet 261 will pass through the interior chamber 257 to the outlet 263 and into the telescoping portion 244.

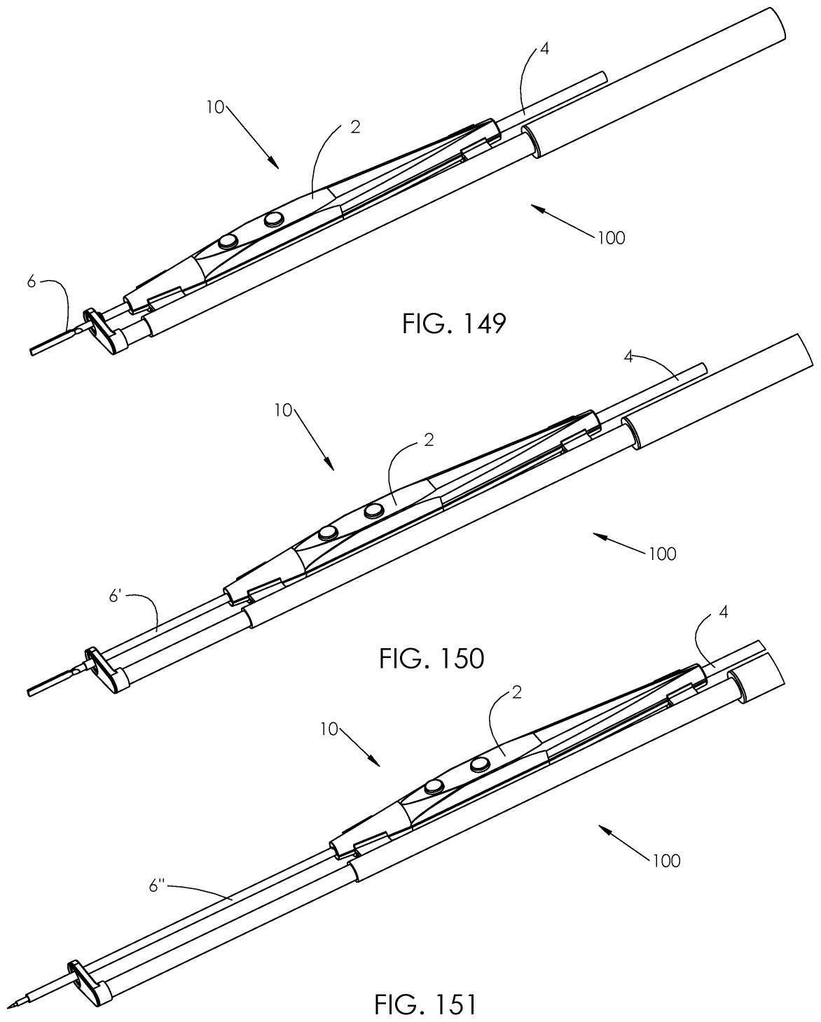

In use, the proximal portion 16 of smoke evacuation device 100 remains fixed relative to the body 2 of instrument 10, while the telescoping portion 244 can be adjusted distally or proximally for optimal positioning to allow for improved suction at the site through nozzle assembly 246 where smoke is generated while providing flexibility to avoid anatomy when in a tight surgical pocket, as well as the ability to match the length of the electrocautery blade/end effector (6, 6′ and 6″), as illustrated in FIGS. 149 through 151.

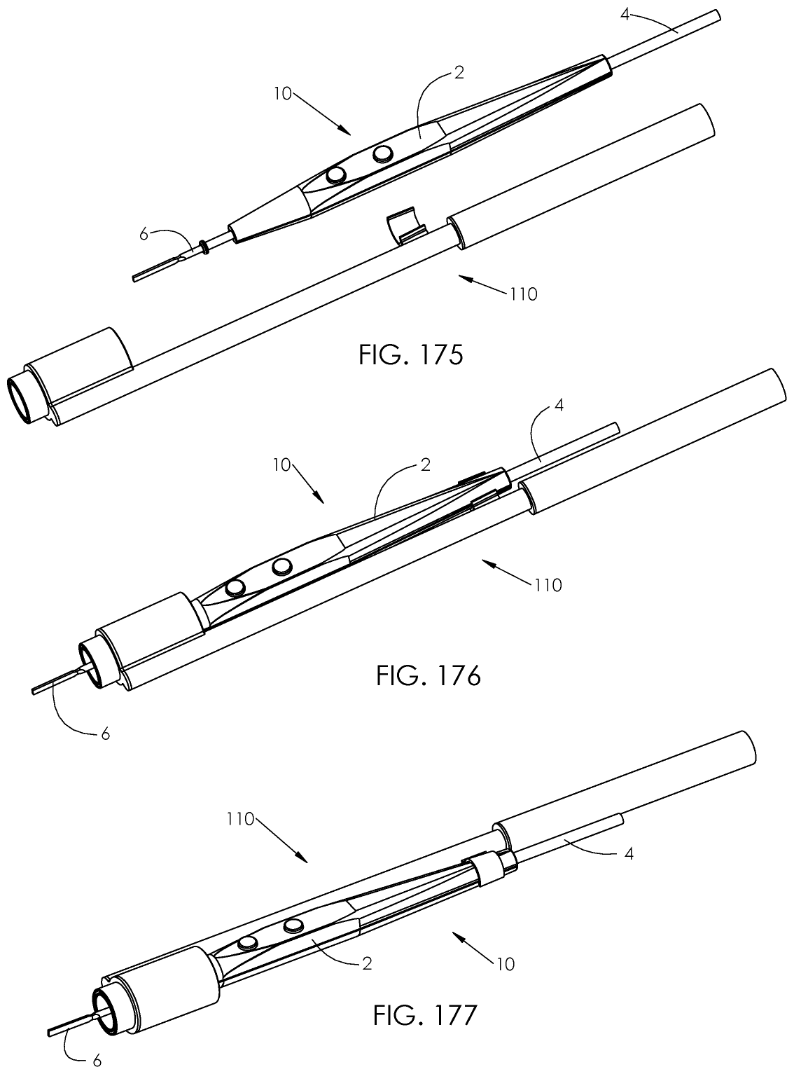

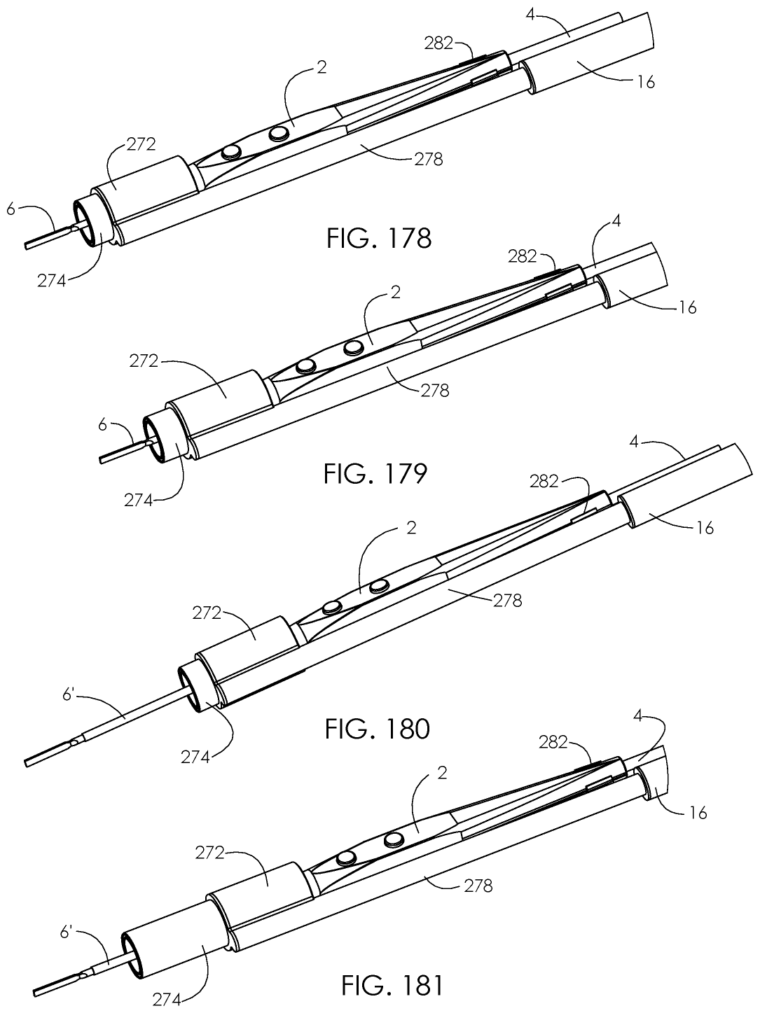

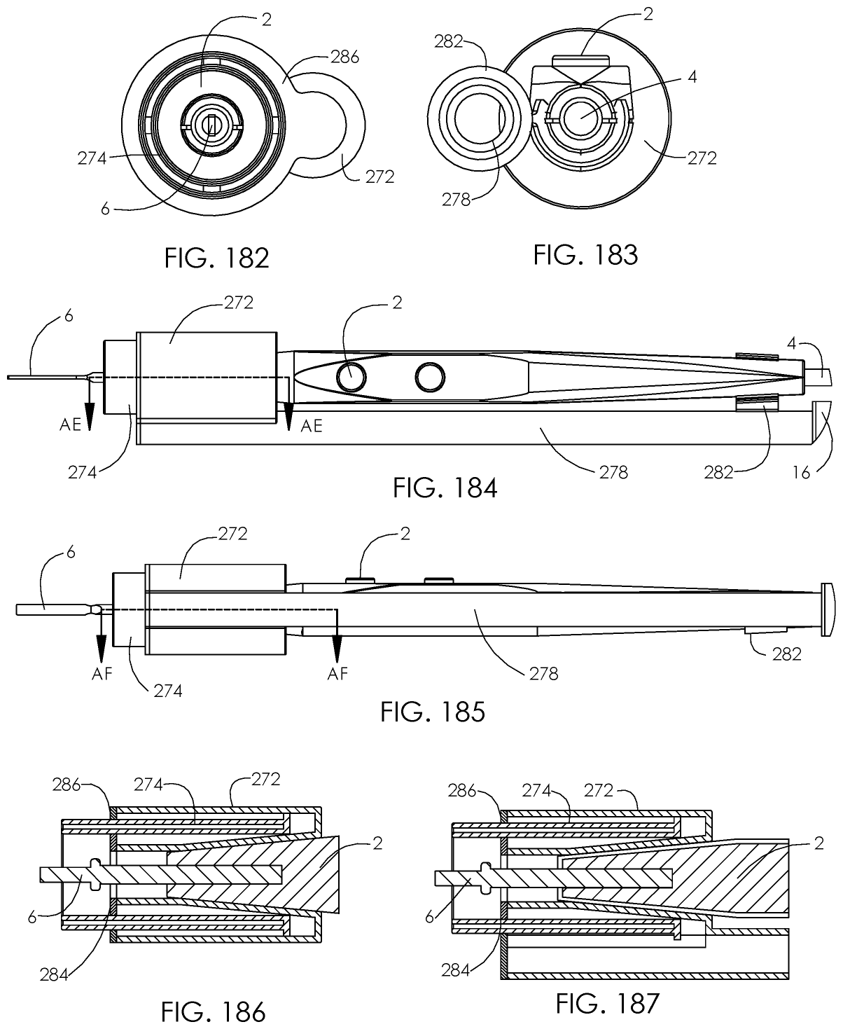

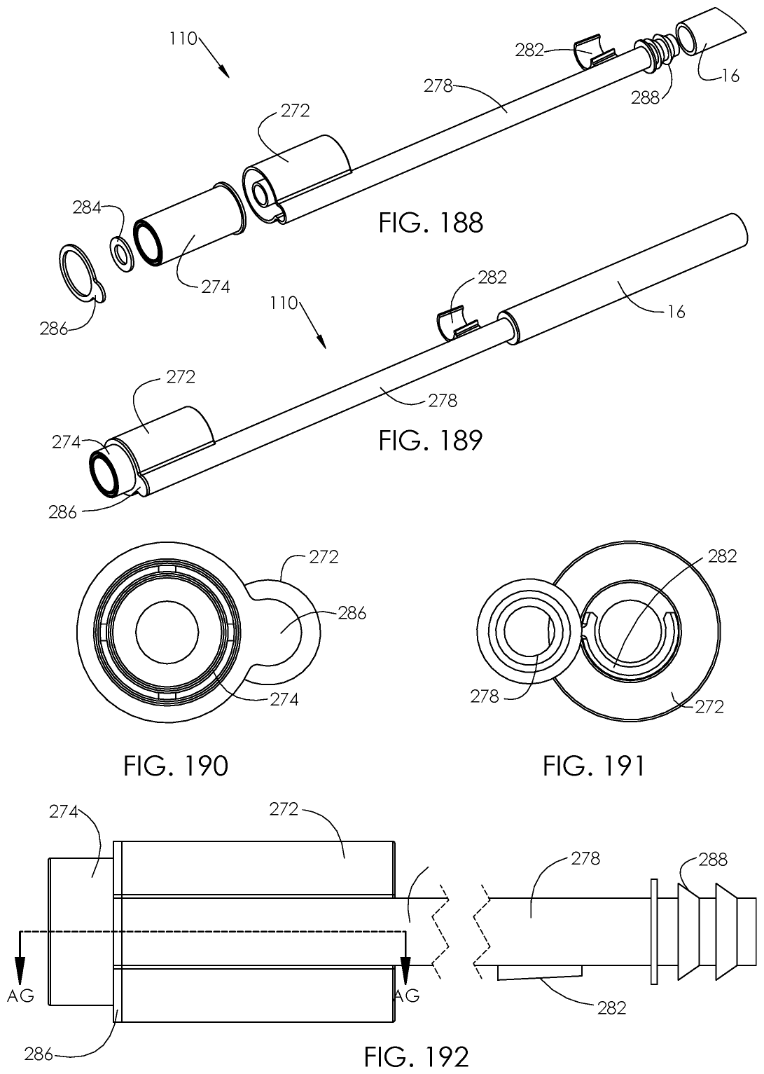

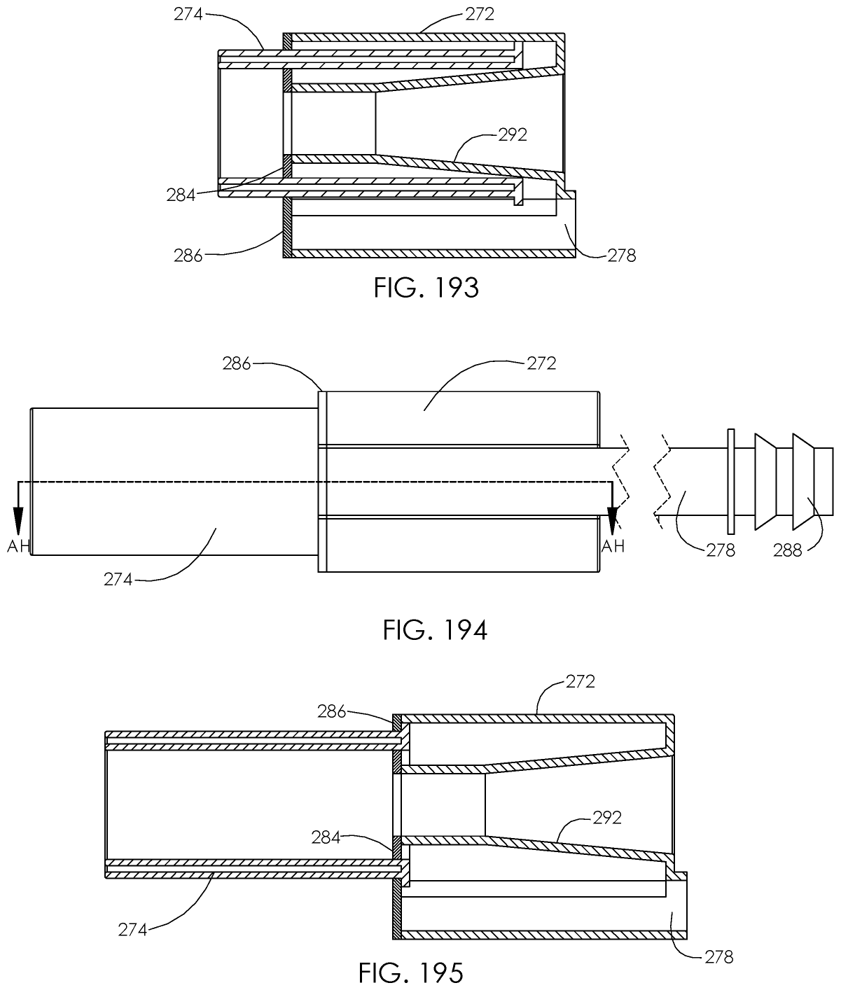

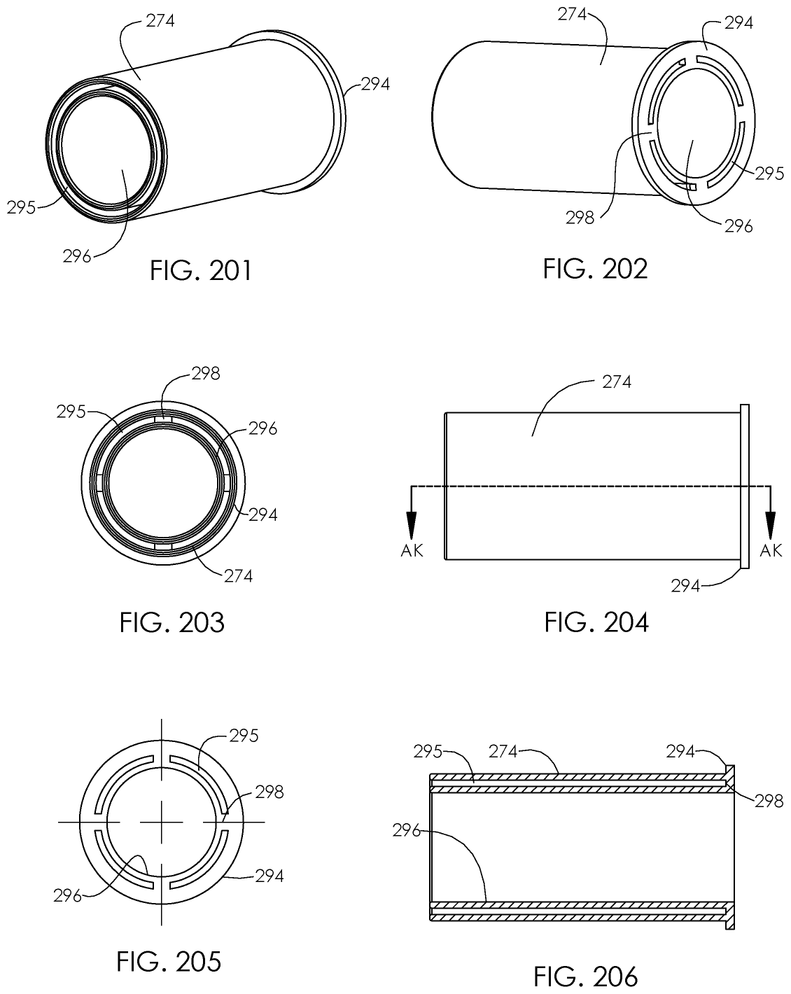

Referring now to FIGS. 175 through 206, there is illustrated another new and useful smoke evacuation device 110 for use with a handheld electrosurgical instrument 10 having an elongated body 2. In this embodiment of the invention, the smoke evacuation device 110 includes a proximal body portion 16 and a distal body portion 278. A sealing barb 288 is provided at the proximal end of distal body portion 278 for engagement within the distal end of proximal body portion 16, as shown in FIG. 194.

Smoke evacuation device 110 differs from the other smoke evacuation devices described herein in that it includes a single attachment feature in the form of a “C” shaped clamp 282 located adjacent the proximal end of distal body portion 278 for clamping on to the proximal end of the body 2 of surgical instrument 10. Instead of a distal attachment feature as provided on each of the other devices described herein, smoke evacuation device 110 includes a distal alignment feature, which also functions as a telescoping nozzle assembly, as described in further detail below.

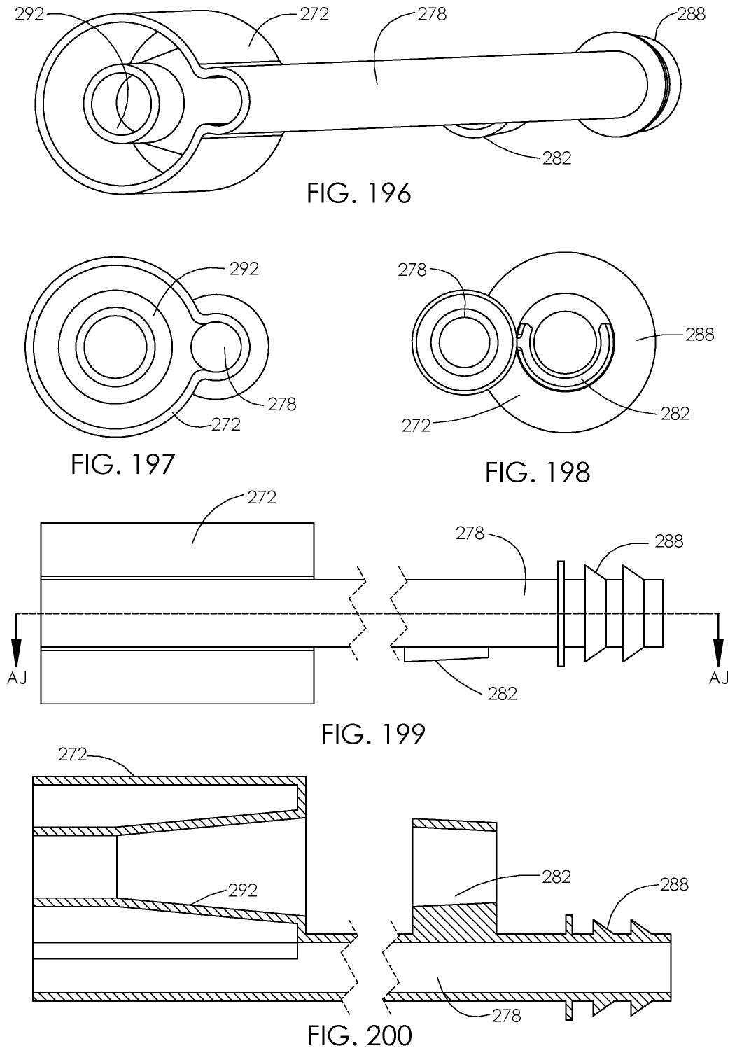

The distal alignment feature includes an outer body portion 272 extending from and fluidly communicating with an inner lateral side of the distal body portion 278, as best seen in FIGS. 196 through 200. The outer body portion 272 includes a conical reception sleeve 292 for receiving the distal end portion of the body 2 of surgical instrument 10, as best seen in FIGS. 184 through 187. The outer body portion 272 further includes an inner telescopic body portion 274 the is slidably associated with the outer body portion 272, so that it an telescopically extends and retracts from the distal end of the outer body portion to allow for improved suction at the site where smoke is generated, as well as the ability to match the length of the electrocautery blade/end effector (6, 6′ and 6″), as illustrated in FIGS. 179 through 181.

The inner body portion 274 further includes an interior sleeve 296 which defines an annular suction channel 295 that extends from the distal end of the inner body portion to a stop flange 294. As best seen in FIG. 202, the stop flange 294 includes a plurality of arcuate ports 298 that facilitate the fluid communication between the annular suction channel 295 and the interior of outer body portion 272, which is in fluid communication with the distal body portion 278. An inside cover 284 is associated with the distal end of the conical reception sleeve 292 and an outside cover 286 is associated with the distal end of the outer body portion 272 for enclosing the interior of the outer body portion 272.

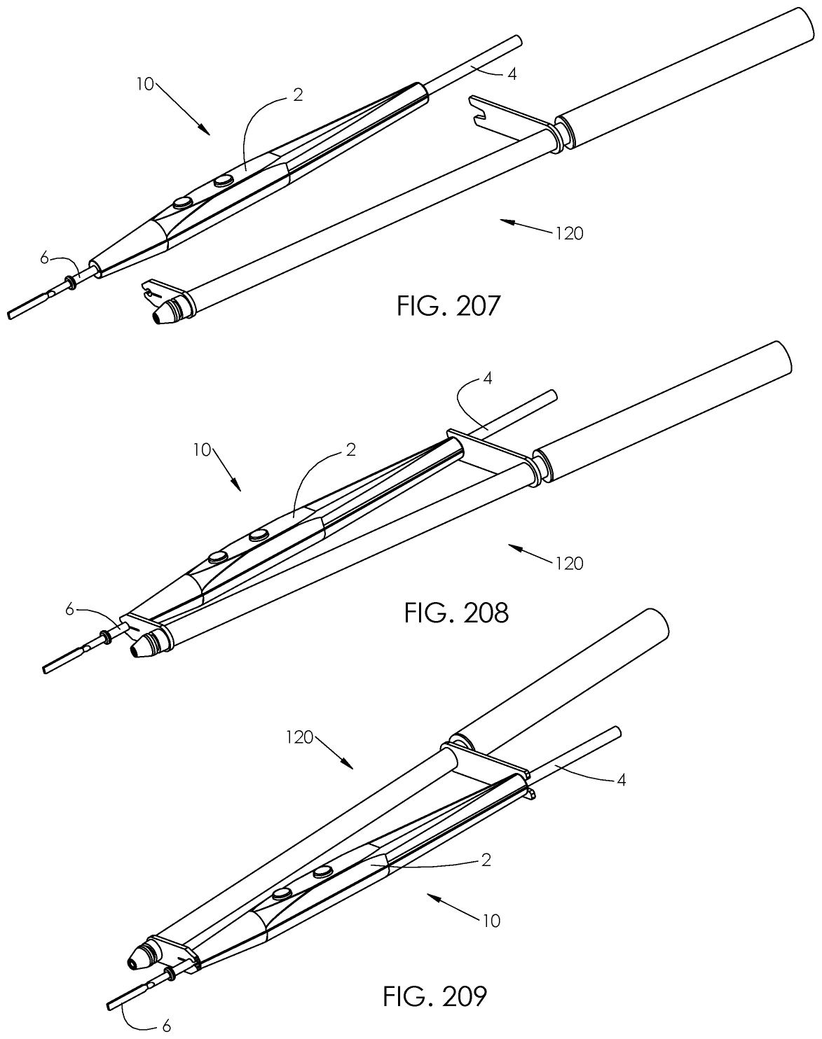

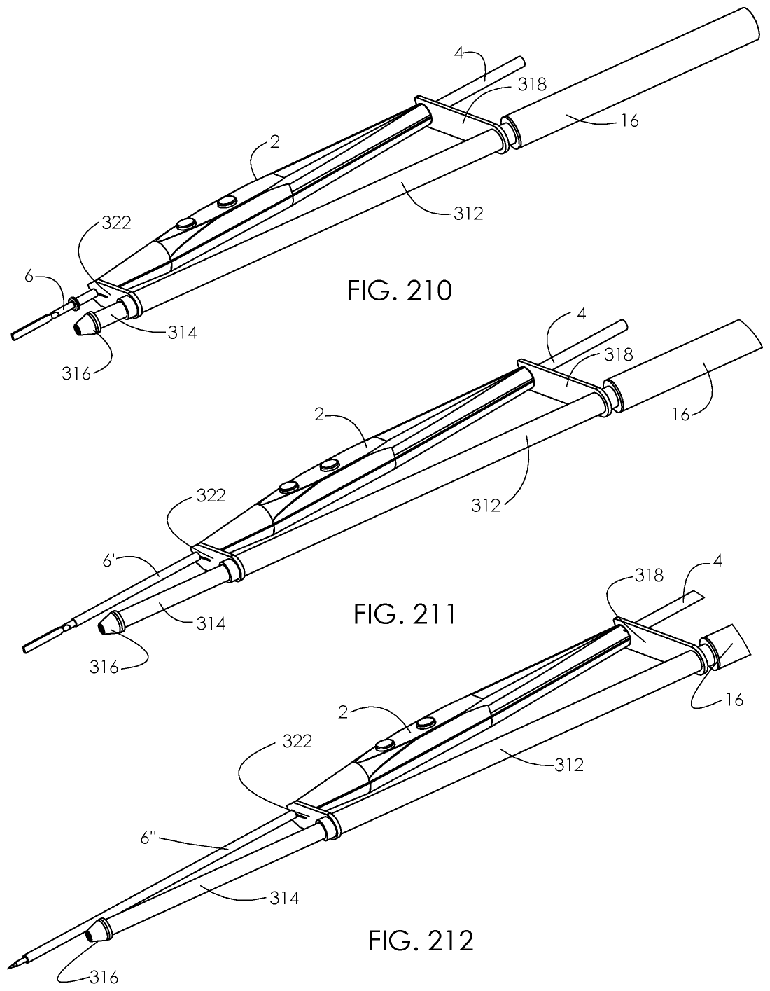

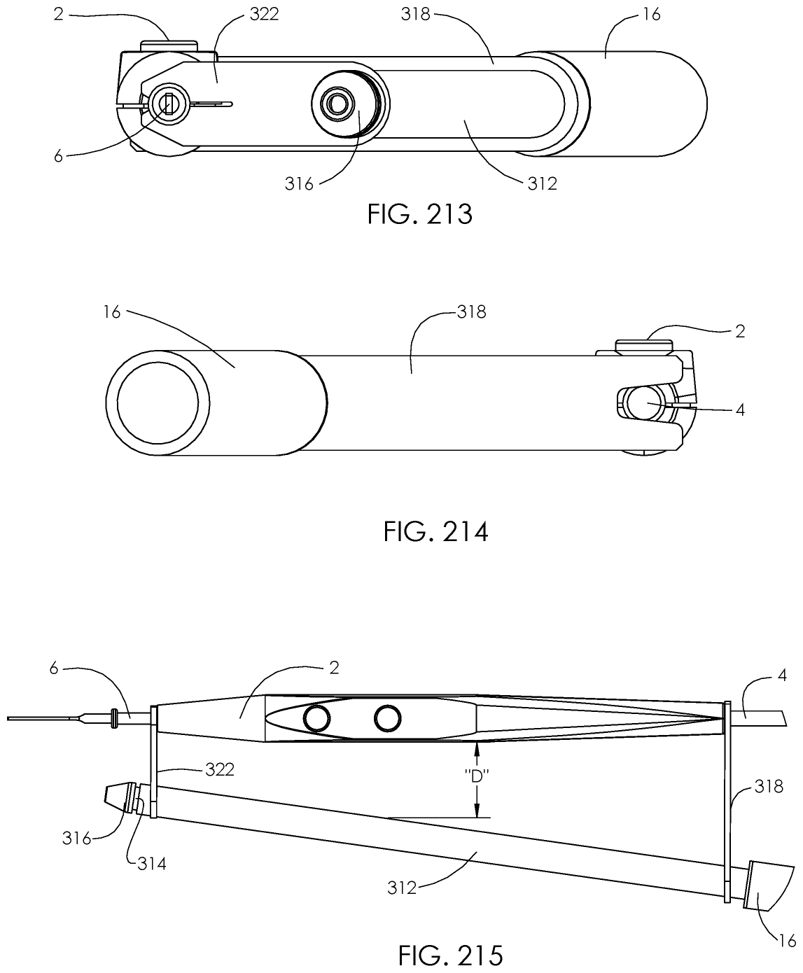

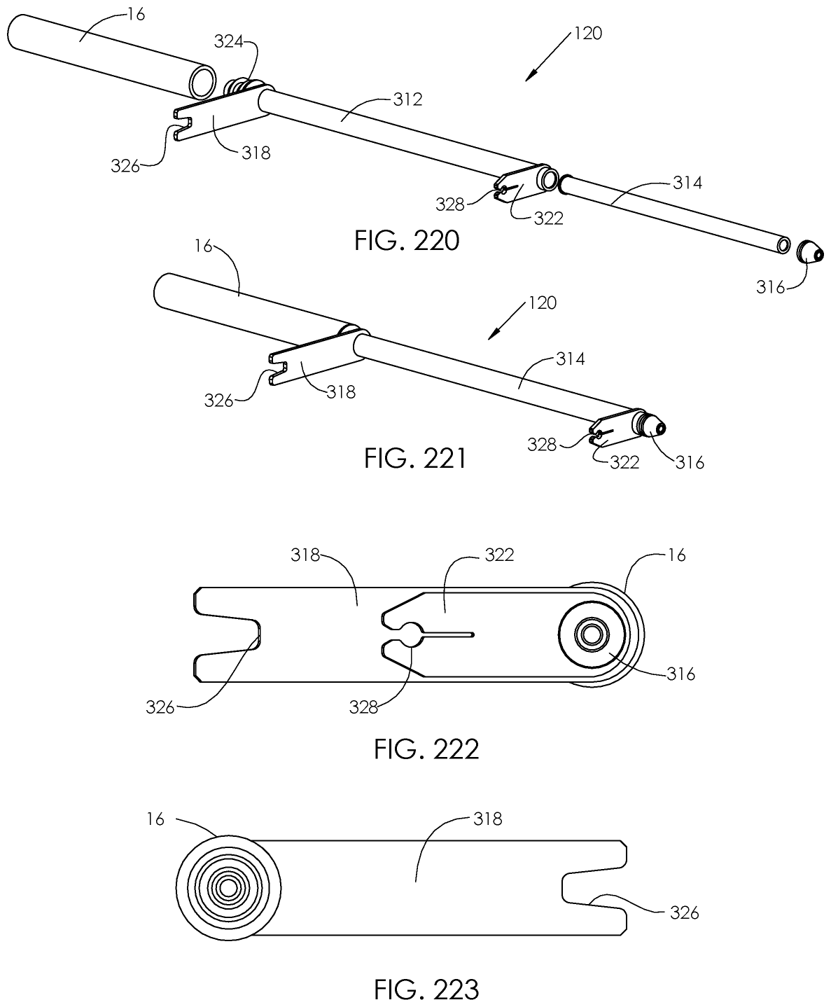

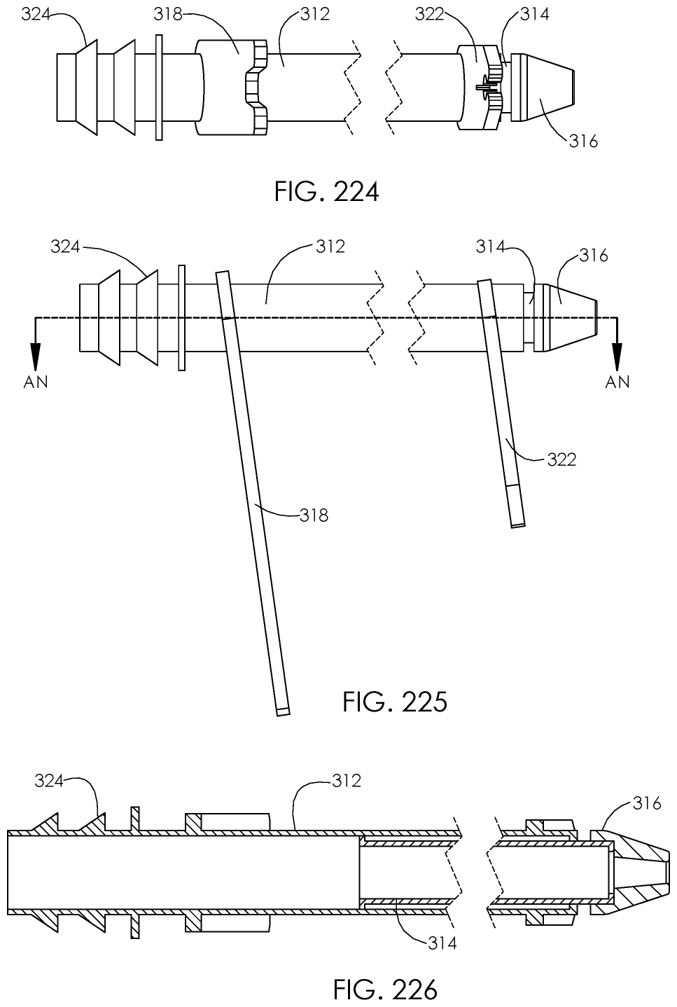

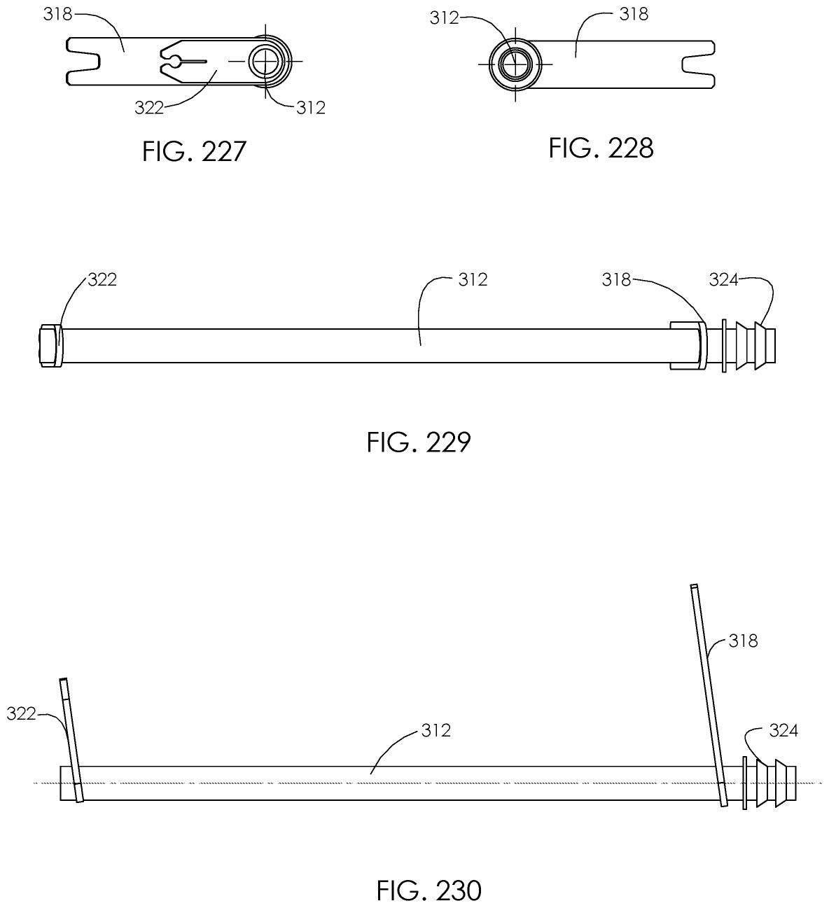

Referring now to FIGS. 207 through 230, there is illustrated another new and useful smoke evacuation device 120 for use with a handheld electrosurgical instrument 10 having an elongated body 2. In this embodiment of the invention, the smoke evacuation device 120 includes a proximal body portion 16, a distal body portion 312 and a telescoping portion 314 having a distal suction tip 316. In use, the telescoping portion 314 can be readily extended and retracted relative to the electrocautery blade/end effector 6 to allow for improved suction at the site where smoke is generated while providing flexibility to avoid anatomy when in a tight surgical pocket, as well as the ability to match the length of the electrocautery blade/end effector, as shown for example in FIGS. 210 through 212, with respect to the three end effectors 6, 6′ and 6″.

A sealing barb 324 is provides at the proximal end of distal body portion 312 for engagement within the distal end of proximal body portion 16, and stop flanges are provided at the proximal end of telescoping portion 324 and the distal end of distal body portion 242, as best seen in FIG. 226.

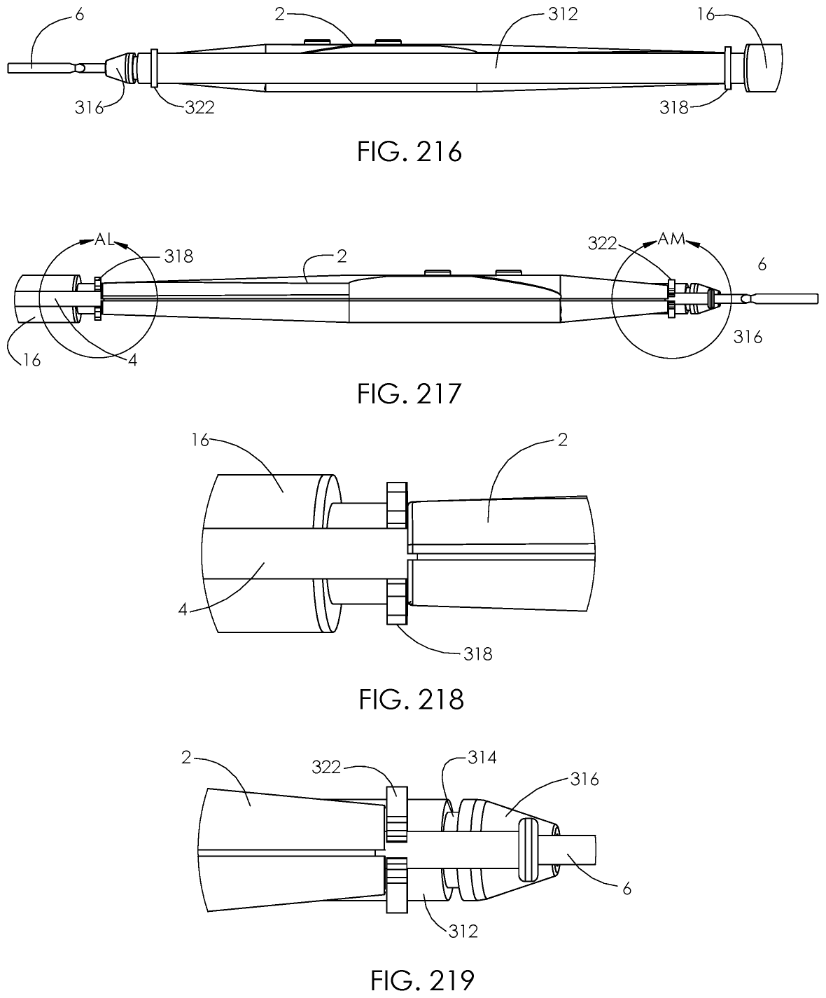

Smoke evacuation device 120 includes a distal attachment feature in the form of a rigid clipping structure 332 supported adjacent a distal end of distal body portion 312 that is adapted and configured to engage the base of the end effector 6 extending of the distal end of surgical instrument 10 and a proximal attachment feature in the form of a rigid clipping structure 318 supported adjacent a proximal end of distal body portion 312 that is adapted and configured to snap onto the proximal end of the body 2 of the surgical instrument 10, as illustrated in FIGS. 216 through 219. The clips 318 and 312 are adapted and configured to enable the smoke evacuation device 120 to be secured to the left or right side of the body 2 of surgical instrument 10,

As best seen in FIG. 215, the length of the distal clipping structure 322 is less than the length of the proximal clipping structure 318. Consequently, a clearance distance “D” is established between the body 2 of surgical instrument 10 and the smoke evacuation device 120, to provide better ergonomics for the user holding the instrument. This allows a surgeon to hold the electrocautery device 10 without interference from the smoke evacuation device 120.

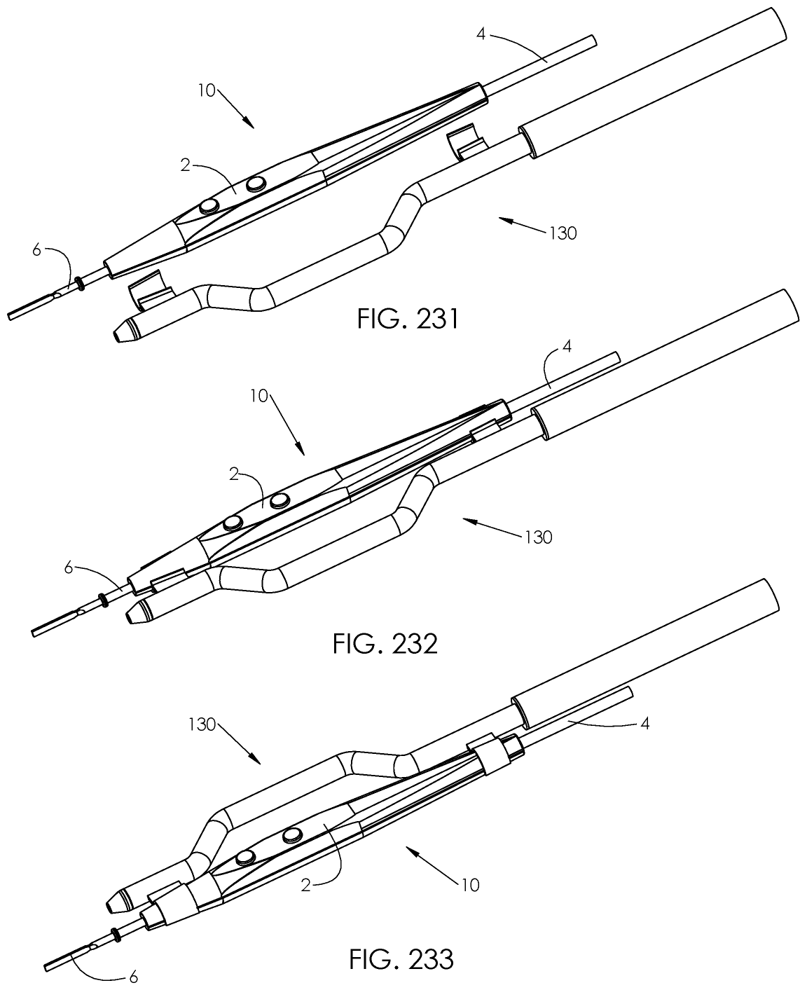

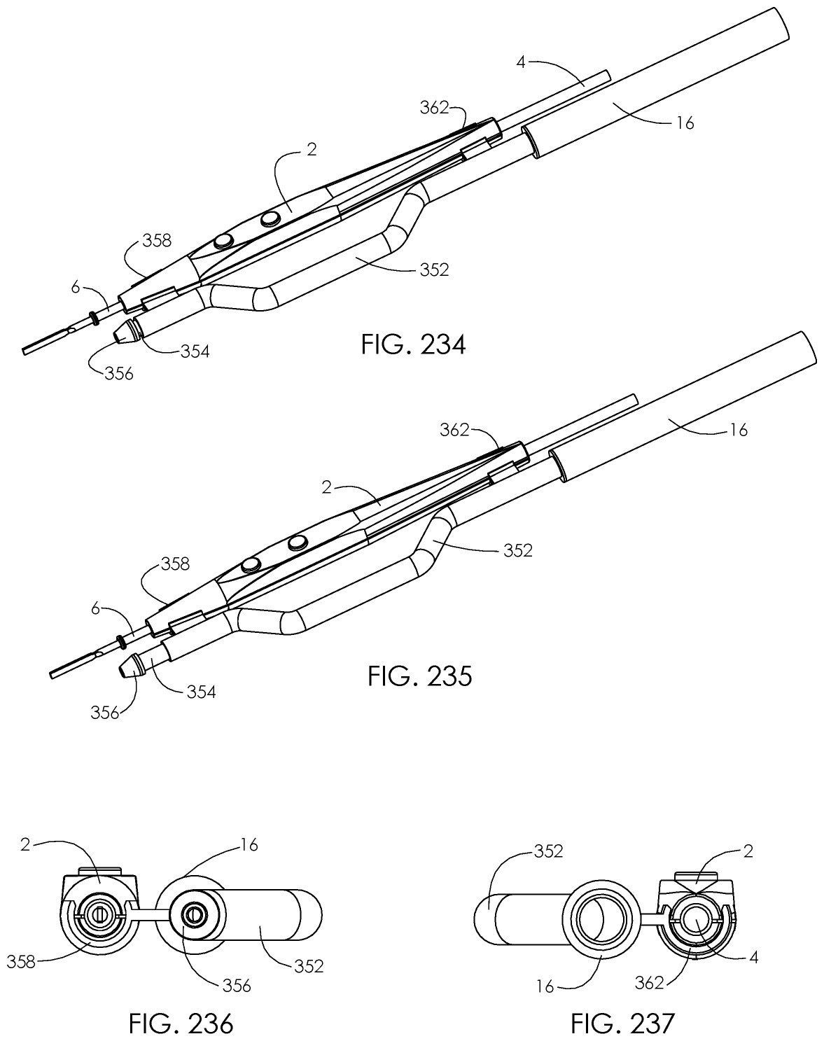

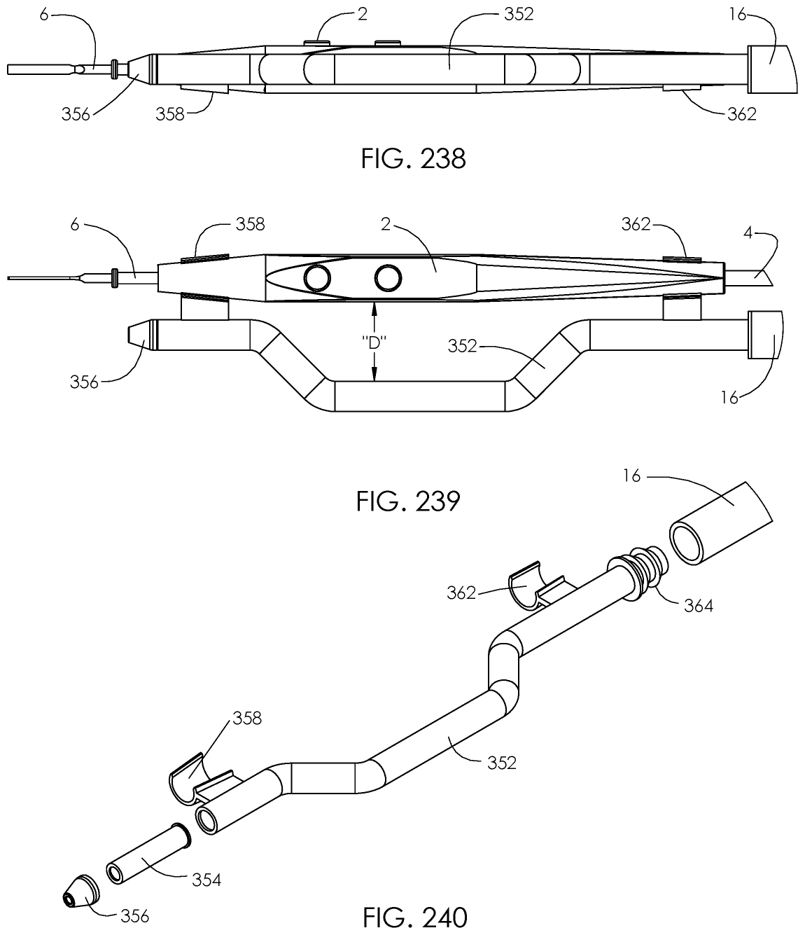

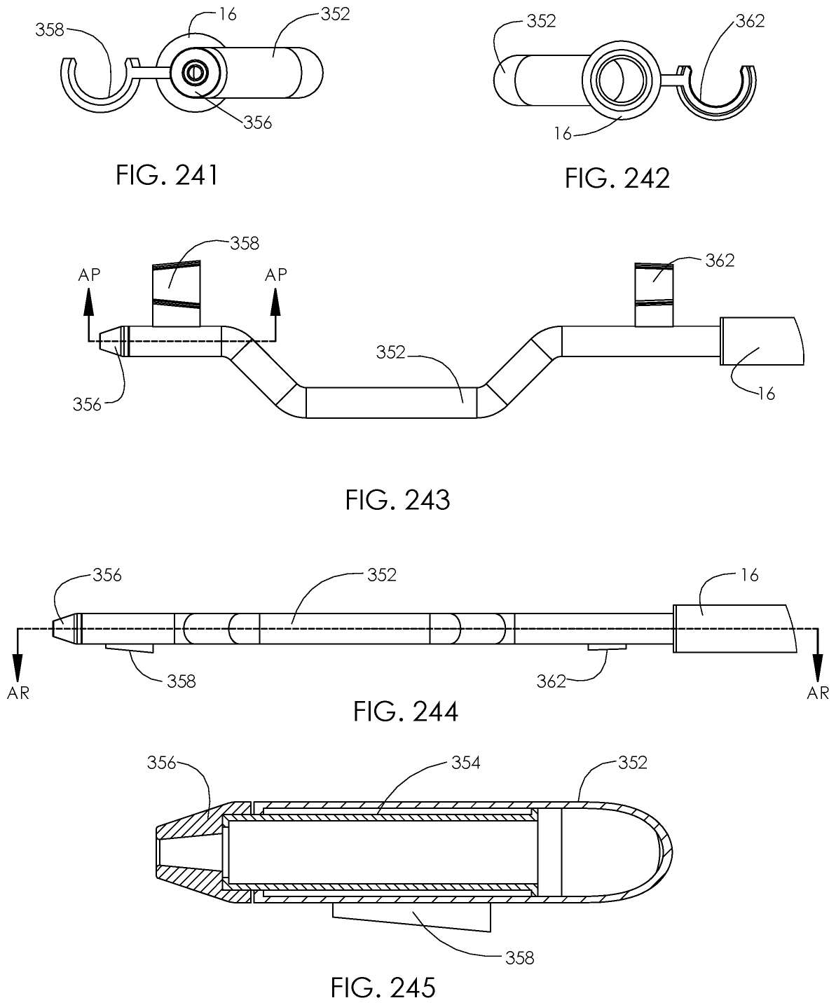

Referring now to FIGS. 231 through 250, there is illustrated another new and useful smoke evacuation device 130 for use with a handheld electrosurgical instrument 10 having an elongated body 2. In this embodiment of the invention, the smoke evacuation device 130 includes a proximal body portion 16, a distal body portion 352 and a telescoping portion 354 with a distal suction tip 356 (see FIGS. 249 and 250). A sealing barb 364 is provides at the proximal end of distal body portion 352 for engagement within the distal end of proximal body portion 16 (see FIG. 247), and stop flanges are provided at the proximal end of telescoping portion 244 and the distal end of distal body portion 242, as best seen in FIG. 248.

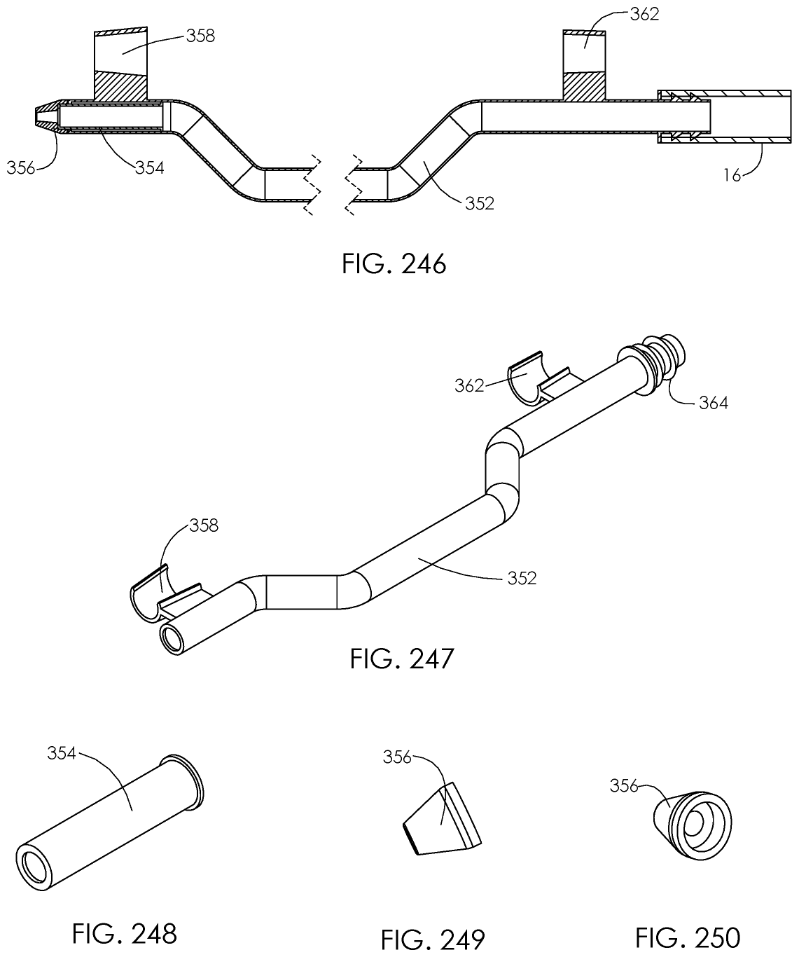

Smoke evacuation device 100 includes a distal attachment feature in the form of a “C” shaped clamp 358 located adjacent the distal end of distal body portion 352 for clamping on to the proximal end of the body 2 of surgical instrument 10. As best seen in FIGS. 246 and 247, clamp 358 has a tapered configuration for accommodating the profile of the distal end portion of body 2 of surgical instrument 10. Smoke evacuation device 100 further includes a proximal attachment feature in the form of another “C” shaped clamp 362 located adjacent the proximal end of distal body portion 352 for clamping on to the proximal end of the body 2 of surgical instrument 10. Clamp 362 has a semi-cylindrical configuration for accommodating the profile of the proximal end portion of body 2 of surgical instrument 10. The clamps 358 and 362 are adapted and configured to enable the smoke evacuation device 130 to be selectively secured to the left or right side of the body 2 of surgical instrument 10, as illustrated in FIGS. 232 and 233.

As best seen in FIG. 239, the distal body portion 352 has an offset curved shaped medial section which is dimensioned and configured to establish a distance “D” between the body 2 of surgical instrument 10 and the smoke evacuation device 130, to provide better ergonomics for the user holding the instrument. In use, the telescoping portion 354 can be readily extended and retracted relative to the electrocautery blade/end effector 6 to allow for improved suction at the site where smoke is generated while providing flexibility to avoid anatomy when in a tight surgical pocket, as well as the ability to match the length of the electrocautery blade/end effector,

While the subject disclosure has been shown and described with reference to preferred embodiments, those skilled in the art will readily appreciate that changes or modifications may be made thereto without departing from the spirit or scope of the subject disclosure.

What is claimed is:

1. A smoke evacuation device for use with a handheld surgical instrument, comprising:
a) an elongated tubular body having opposed proximal and distal end portions, wherein the proximal end portion of the tubular body is adapted and configured for communicating with a source of suction and the distal end portion of the tubular body is adapted and configured to intake smoke generated at a surgical site; and
b) distal attachment means for attaching the distal end portion of the tubular body adjacent to a distal end portion of the handheld surgical instrument.

2. A smoke evacuation device as recited in claim 1, wherein the distal attachment means includes a strap of adhesive material, a hook and loop type fastener strap or a cable tie strap.

3. A smoke evacuation device as recited in claim 1, wherein the distal attachment means includes a spring biased compression clamp with compliant contact surfaces.

4. A smoke evacuation device as recited in claim 1, wherein the distal attachment means includes a pair of spaced apart deflectable arms with compliant contact surfaces.

5. A smoke evacuation device as recited in claim 1, wherein the distal attachment means includes a flexible snap-on curved compression clasp.

6. A smoke evacuation device as recited in claim 1, wherein the distal attachment means includes a mounting block adapted and configured for spring biased compressive engagement on the distal end portion of the handheld surgical instrument, which includes a rail on a side wall thereof for slidably engaging with an elongated track extending along the tubular body.

7. A smoke evacuation device as recited in claim 6, wherein a pinion gear is included on the side wall of the mounting block for cooperating with a gear rack provided adjacent the elongated track for advancing or retracting the tubular body relative to the distal end portion of the handheld surgical instrument.

8. A smoke evacuation device as recited in claim 1, wherein the distal attachment means is an alignment feature that includes a mounting sleeve adapted and configured for surrounding engagement on the distal end portion of the handheld surgical instrument, the mounting sleeve including an outer body portion and an inner body portion that is mounted to extend from and retract into the outer body portion to accommodate end effectors of differing length.

9. A smoke evacuation device as recited in claim 8, wherein an annular smoke evacuation passage is formed between the outer body portion and the inner body portion of the mounting sleeve.

10. A smoke evacuation device as recited in claim 1, wherein the distal attachment means includes a clip for engaging an end effector extending from the distal end portion of the handheld surgical instrument.

11. A smoke evacuation device as recited in claim 1, further comprising proximal attachment means for attaching the proximal end portion of the tubular body adjacent to a proximal end portion of the handheld surgical instrument.

12. A smoke evacuation device as recited in claim 11, wherein the proximal attachment means includes a clip for engaging a power cord extending from the proximal end portion of the handheld surgical instrument.

13. A smoke evacuation device as recited in claim 10, wherein the proximal attachment means incudes a pair of spaced apart deflectable arms with compliant contact surfaces.

14. A smoke evacuation device as recited in claim 10, wherein the proximal attachment means includes a flexible snap-on curved compression clasp.

15. A smoke evacuation device as recited in claim 1, wherein the tubular body includes a telescoping tube adapted and configured to extend from and retract into the distal end of the tubular body.

16. A smoke evacuation device as recited in claim 15, wherein the telescoping tube and the tubular body are threadably associated with one another.

17. A smoke evacuation device as recited in claim 15, wherein a proximal end of the telescoping tube and the distal end of the tubular body are sealingly associated with one another.

18. A smoke evacuation device as recited in claim 1, wherein the tubular body has an offset section between the opposed proximal and distal end portions thereof to provide a predetermined ergonomic distance between the tubular body and the handheld surgical instrument.

19. A smoke evacuation device as recited in claim 15, wherein a nozzle tip is operatively associated with a distal end of the telescoping tube.

20. A smoke evacuation device as recited in claim 19, wherein the nozzle tip has a tapered conical construction.

21. A smoke evacuation device as recited in claim 19, wherein the nozzle tip has an intake portion that surrounds an end effector extending from the distal end portion of the handheld surgical instrument.

22. A smoke evacuation device as recited in claim 1, wherein the distal attachment means is adapted and configured to maintain a predetermined ergonomic distance between the tubular body and the handheld surgical instrument.

23. A smoke evacuation device for use with a handheld electrosurgical instrument, comprising:
a) an elongated tubular body having opposed proximal and distal end portions, wherein the proximal end portion of the tubular body is adapted and configured for communicating with a source of suction and the distal end portion of the tubular body is adapted and configured to intake smoke generated at a surgical site by the handheld electrosurgical instrument;
b) distal attachment means for attaching the distal end portion of the tubular body adjacent to a distal end portion of the handheld surgical instrument; and
c) proximal attachment means for attaching the proximal end portion of the tubular body adjacent to a proximal end portion of the handheld surgical instrument.

24. A smoke evacuation device as recited in claim 23, wherein the tubular body includes at least one telescoping tube adapted and configured to extend from and retract into the distal end of the tubular body to allow for improved suction at the surgical site where smoke is generated.

25. A smoke evacuation device as recited in claim 23, wherein the distal and proximal attachment means are adapted and configured to maintain a predetermined ergonomic distance between the tubular body and the handheld surgical instrument.

26. A smoke evacuation device for use with a handheld surgical instrument, comprising:
a) an elongated tubular body having opposed proximal and distal end portions, wherein the proximal end portion of the tubular body is adapted and configured for communicating with a source of suction and the distal end portion of the tubular body is adapted and configured to intake smoke generated at a surgical site; and
b) proximal attachment means for attaching the proximal end portion of the tubular body adjacent to a proximal end portion of the handheld surgical instrument.

27. A smoke evacuation device as recited in claim 26, further comprising a distal alignment feature that includes a mounting sleeve adapted and configured for surrounding engagement on the distal end portion of the handheld surgical instrument.

28. A smoke evacuation device as recited in claim 27, wherein the mounting sleeve includes an outer body portion and an inner body portion, and wherein the inner body portion is mounted to extend from and retract into the outer body portion to accommodate end effectors of differing length.

29. A smoke evacuation device as recited in claim 28, wherein an annular smoke evacuation passage is formed between the outer body portion and the inner body portion of the mounting sleeve.

30. A smoke evacuation device as recited in claim 26, wherein the proximal attachment means includes a flexible compression clasp.
